(19) United States
(12) Patent Application Publication
(10) Pub. No.: US 2006/0167030 A1
(43) Pub. Date: Jul. 27, 2006

(54) Sulfonamido-macrocycles and the salts thereof, a pharmaceutical composition comprising these compounds, the method of preparing and the use thereof

(76) Inventors: Georg Kettschau, Berlin (DE); Hans Briem, Bremen (DE); Andreas Huth, Berlin (DE); Ulrich Luecking, Berlin (DE); Martina Schaefer, Berlin (DE); Karl-Heinz Thierauch, Berlin (DE); Wolfgang Schwede, Glienicke (DE); Manfred Husemann, Hohen Neuendorf (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/312,646

(22) Filed: Dec. 21, 2005

Related U.S. Application Data
(60) Provisional application No. 60/639,313, filed on Dec. 28, 2004.

(30) Foreign Application Priority Data
Dec. 22, 2004 (DE) 0409509.3

(51) Int. Cl. A61K 31/519; C07D 498/04

(52) U.S. Cl. 514/267; 540/468

(57) ABSTRACT

The invention relates to sulfonamido-macrocycles according to the general Formula I and the salts thereof, to pharmaceutical compositions comprising the sulfonamido-macrocycles and to a method of preparing the sulfonamido-macrocycles as well as the use thereof for manufacturing a pharmaceutical composition for the treatment of diseases of dysregulated vascular growth or of diseases which are accompanied with dysregulated vascular growth, wherein the compounds effectively interfere with angiopoietin and therefore influence Tie2 signalling. 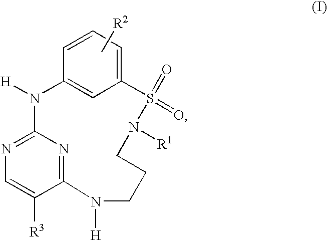
wherein R¹, R² and R³ have the meaning as given in the specification and the claims.

This application claims the benefit of the filing date of U.S. Provisional Application Ser. No. 60/639,313 filed Dec. 28, 2004 which is incorporated by reference herein.

The invention relates to sulfonamido-macrocycles and the salts thereof, to pharmaceutical compositions comprising the sulfonamido-macrocycles and to methods of preparing the sulfonamido-macrocycles as well as to the use thereof.

In order to defeat diseases with dysregulated vascular growth such as cancer different strategies were developed. One possible strategy is the blockade of angiogenesis to the tumour tissue, because tumour angiogenesis is a prerequisite for the growth of solid tumours.

The angiogenesis represents beside the vasculogenesis one of two basic processes during the genesis of vasculature. Vasculogenesis names the neoplasm of vasculature during the embryo development, wherein the angiogenesis describes the neoplasm of vasculature by sprouts or division of present vasculature. It has been found that two receptors expressed on endothelial cells, VEGF—(vascular endothelial growth factor) and Tie-receptors, are essential for normal development of vascular tissue as blood vessels (Dumont et al., (1994). Dominant-negative and targeted null mutations in the endothelial receptor tyrosine kinase Tie2 (also named tek) reveal a critical role in vasculogenesis of the embryo. Genes Dev, 8:1897-909; Sato et al.: “Distinct roles of the receptor tyrosine kinases Tie-1 and Tie-2 in blood vessel formation” Nature. 1995, July 6; 376(6535):70-4.).

The mechanism of Tie2 signalling was characterized by different researchers, wherein different angiopoietins were found to be involved. So it could be explained that angiopoietin-1 if bound to the extracellular domain of the Tie2-receptor stimulates autophosphorylation and activates the intracellular kinase domain. Angiopoietin-1 activation of Tie2 however does not stimulate mitogenesis but rather migration. Angiopoietin-2 can block angiopoietin-1 mediated Tie2 activation and the resulting endothelial migration. This indicates that angiopoietin-2 is a naturally occurring inhibitor of Tie2 activation (Maisonpierre et al.: “Angiopoietin-2, a natural antagonist for Tie2 that disrupts in vivo angiogenesis”. Science. 1997, July 4; 277(5322):55-60; WitzenbichLer et al.: “Chemotactic properties of angiopoietin-1 and -2, ligands for the endothelial-specific receptor tyrosine kinase Tie2”. J Biol Chem. 1998, July 17; 273(29):18514-21).

### BRIEF DESCRIPTION OF THE DRAWINGS

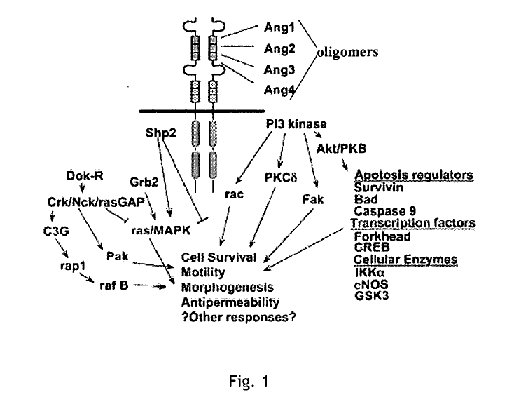

For an overview see FIG. 1 represents an overview of Tie2 signalling, modified by Peters et al. (Peters et al.: “Functional significance of Tie2 signalling in the adult vasculature”. Recent Prog Horm Res. 2004; 59:51-71. Review.).

Receptor dimerization results in cross-phosphorylation on specific tyrosine-residues. Receptor cross-phosphorylation has a dual effect: it enhances the receptor's kinase activity and it provides binding sites for signalling molecules possessing phosphotyrosine binding domains (SH2 and PTB domains) (Pawson T.: “Regulation and targets of receptor tyrosine kinases”. Eur J Cancer. 2002, September, 38 Suppl 5:S3-10. Review).

The signalling cross-talk between the PI3-K pathway and the Dok-R pathway is required for an optimal chemotactic response downstream of Tie2. Other recent studies have shown that Tie2-mediated activation of the PI3-K/Akt pathway is required for endothelial nitric oxide synthase (eNOS) activation, focal adhesion kinase activation, and protease secretion, all of which may contribute importantly to Tie2 function during angiogenesis (Kim I. et al.: “Angiopoietin-1 regulates endothelial cell survival through the phosphatidylinositol 3′-Kinase/Akt signal transduction pathway”. Circ Res. 2000, January 7-21; 86(1):24-9; Babaei et al.: “Angiogenic actions of angiopoietin-1 require endothelium-derived nitric oxide”. Am J Pathol. 2003, June; 162(6):1927-36).

For normal development a balanced interaction between the receptors and so-called ligands is necessary. Especially the angiopoietins, which signal via Tie2 receptors, play an important role in angiogenesis (Babaei et al., 2003).

The broad expression of Tie2 in adult vasculature has been confirmed in transgenic mice using Tie2 promoter driven reporters (Schlaeger et al.: “Uniform vascular-endothelial-cell-specific gene expression in both embryonic and adult transgenic mice”. Proc Natl Acad Sci USA. 1997, April 1; 94(7):3058-63; Motoike et al.: “Universal GFP reporter for the study of vascular development”. Genesis. 2000, October; 28(2):75-81). Immunohistochemical analysis demonstrated the expression of Tie2 in adult rat tissues undergoing angiogenesis. During ovarian folliculogenesis, Tie2 was expressed in the neo-vessels of the developing corpus Luteum. Angiopoietin-1 and angiopoietin-2 also were expressed in the corpus luteum, with angiopoietin-2 localizing to the leading edge of proliferating vessels and angiopoietin-1 localizing diffusely behind the leading edge (Maisonpierre et al., 1997). It was suggested that angiopoietin-2-mediated inhibition of Tie2 activation serves to “destabilize” the vessel, to make it responsive to other angiogenic growth factors such as VEGF. Subsequently, angiopoietin-1-mediated activation of Tie2 would trigger stabilization of the neovasculature. The disruption of Tie2 function shows the relevance of Tie2 for neoangiogenesis in transgenic mice resulting in early embryonic lethality as a consequence of vascular abnormalities (Dumont et al., 1994; Sato et al., 1995). Tie2−/− embryos failed to develop the normal vessel hierarchy, suggestive of a failure of vascular branching and differentiation. Tie2−/− embryos have a decreased number of endothelial cells and furthermore less contact between endothelial cells and the underlying pericytes/smooth muscle cells. This implies a role in the maturation and stabilization of newly formed vasculature.

The studies in mice with transgenic or ablated Tie2 gene suggest a critical role for Tie2 in maturation of vascular development in embryos and in adult vasculature. Conditional expression of Tie2 in the endothelium of mice homozygous for a Tie2 null allele partially rescued the embryonic lethality of the Tie2 null phenotype (Jones N et al.: “Tie receptors: new modulators of angiogenic and lymphangiogenic responses.” Nat Rev Mol Cell Biol. 2001 April; 2(4):257-67. Review). Mice lacking functional angiopoietin-1 expression and mice over-expressing angiopoietin-2 both displayed a phenotype similar to Tie2−/− mice (Suri et al.: “Requisite role of angiopoietin-1, a ligand for the Tie2 receptor, during embryonic angiogenesis.” Cell. 1996 Dec. 27; 87(7): 1171-80; Maisonpierre PC et al.: “Angiopoietin-2, a natural antagonist for Tie2 that disrupts in vivo angiogenesis. Science. 1997 July 4; 277(5322):55-60.).

Angiopoietin-2−/− mice have profound defects in the growth and patterning of lymphatic vasculature and fail to remodel and regress the hyaloid vasculature of the neonatal lens (Gale et al.: “Angiopoietin 2 is required for postnatal angiogenesis and lymphatic patterning, and only the latter role is rescued by Angiopoietin-1”. Dev Cell. 2002, September; 3(3):411-23). Angiopoietin-1 rescued the lymphatic defects, but not the vascular remodelling defects. So angiopoietin-2 might function as a Tie2 antagonist in blood vasculature but as a Tie2 agonist in developing lymph vasculature.

Tie2 also plays a role in pathological angiogenesis. It was shown that mutations in Tie2 cause inherited venous malformations and enhance both Ligand dependent and independent Tie2 kinase activity (Vikkula et al.: “Dysmorphogenesis caused by an activating mutation in the receptor tyrosine kinase Tie2”. Cell. 1996, December 27; 87(7):1181-90). Tie2 expression was investigated in human breast cancer tumour specimens and Tie2 expression was found in the vascular endothelium both in normal breast tissue and in breast tumours. The proportion of Tie2-positive tumour microvessels was increased in tumours as compared to normal breast tissue (Peters K G et al.: “Expression of Tie2/Tek in breast tumour vasculature provides a new marker for evaluation of tumour angiogenesis. Br J Cancer. 1998, 77(1):51-6).

Angiopoietin-1 overexpression in tumour models resulted in decreased tumour growth. The effect is possibly related to angiopoietin-1 mediated stabilization of the tumour vasculature, which renders the vessels resistant to angiogenic stimuli (Hayes et al.: “Expression and function of angiopoietin-1 in breast cancer”. Br J Cancer. 2000, November; 83(9):1154-60; Shim et al.: “Inhibition of angiopoietin-1 expression in tumour cells by an antisense RNA approach inhibited xenograft tumour growth in immunodeficient mice”. Int J Cancer. 2001, October 1; 94(1):6-15; Shim et al.: “Angiopoietin 1 promotes tumour angiogenesis and tumour vessel plasticity of human cervical cancer in mice”. Exp Cell Res. 2002, October 1; 279(2):299-309; Hawighorst et al.: “Activation of the Tie2 receptor by angiopoietin-1 enhances tumour vessel maturation and impairs squamous cell carcinoma growth”. Am J Pathol. 2002, April; 160(4):1381-92.; Stoeltzing et al.: “Angiopoietin-1 inhibits vascular permeability, angiogenesis, and growth of hepatic colon cancer tumours”. Cancer Res. 2003, June 15; 63(12):3370-7.).

Corneal angiogenesis induced by tumour cell conditioned medium was inhibited by recombinant sTie, despite the presence of VEGF. Mammary tumour growth was significantly inhibited in a skin chamber tumour model by recombinant sTie2 (Lin et al.: “Inhibition of tumour angiogenesis using a soluble receptor establishes a role for Tie2 in pathologic vascular growth”. J Clin Invest. 1997, October 15;100(8):2072-8; Lin et al.: “Antiangiogenic gene therapy targeting the endothelium-specific receptor tyrosine kinase Tie2”. Proc Natl Acad Sci USA. 1998, July 21; 95(15):8829-34). Similar sTie constructs have shown comparable effects in different tumour models (Siemeister et al.: “Two independent mechanisms essential for tumour angiogenesis: inhibition of human melanoma xenograft growth by interfering with either the vascular endothelial growth factor receptor pathway or the Tie-2 pathway”. Cancer Res. 1999, July 1; 59(13):3185-91; Stratmann et al.: “Differential inhibition of tumour angiogenesis by Tie2 and vascular endothelial growth factor receptor-2 dominant-negative receptor mutants”. Int J Cancer. 2001, February 1; 91 (3):273-82; Tanaka et al.: “Tie2 vascular endothelial receptor expression and function in hepatocellular carcinoma”. Hepatology. 2002, April; 35(4):861-7).

When the interaction of angiopoietin-2 with its receptor is blocked by application of a neutralizing anti-angiopoietin-2 monoclonal antibody, the growth of experimental tumours can be blocked efficiently again pointing to the important role of Tie2 in tumour angiogenesis and growth (Oliner et al.: “Suppression of angiogenesis and tumour growth by selective inhibition of angiopoietin-2”. Cancer Cell. 2004, November; 6(5):507-16.) So inhibiting the Tie2 pathway will inhibit pathological angiogenesis.

To influence the interaction between receptor and ligand it could be shown that angiogenesis may be blocked with blockers such as Avastin which interfere with VEGF signal transduction to endothelial cells.

Avastin is a clinically effective antibody that functions as tumour growth inhibitor by blockade of VEGFR mediated angiogenic signalling. Thus interference with VEGF signalling is a proven clinical principle. VEGF-C is a molecule inducing lymph angiogenesis via VEGFR 3. The blockade of this signal pathway is inhibiting diseases associated with lymph angiogenesis as is lymphedema and related diseases (Saharinen et al.: “Lymphatic vasculature: development, molecular regulation and role in tumour metastasis and inflammation.” Trends Immunol. 2004, July:25(7): 387-95. Review).

Pyrimidines and their derivatives have been frequently described as therapeutic agents for diverse diseases. A series of recently published patent applications describes their use as inhibitors of various protein kinases, for example WO 2003/032994 A, WO 2003/063794 A, and WO 2002/096888 A. More specifically, certain pyrimidine derivatives have been disclosed as inhibitors of protein kinases involved in angiogenesis, such as VEGF or Tie2, for example benzimidazole substituted 2,4-diaminopyrimidines (WO 2003/074515 A) or (bis)anilino-pyrimidines (WO 2003/066601 A). Very recently, pyrimidine derivatives in which the pyrimidine constitutes a part of a macrocyclic ring system have been reported to be inhibitors of CDKs and/or VEGF (WO 2004/026881 A), or of CDK2 and/or CDK5, respectively (WO 2004/078682 A).

A particular problem in using such known substances as inhibitors or blockers is that their use at the same time is often accompanied with undesired cytotoxic side effects on normal developing and proliferating tissue. This originates from substances which are less selective and at the same time dose tolerability problems.

Therefore the aim of the present invention is to provide compounds, which are useful for the treatment of diseases of dysregulated vascular growth or diseases which are accompanied by dysregulated vascular growth. Furthermore the prior art problems shall be prevented, especially compounds shall be provided, which show low toxic side effects on normal proliferating tissue but are effectively inhibiting endothelial cell migration at small concentrations. This will further reduce undesired side effects.

The solution to the above problems is achieved by providing compounds derived from a class of sulfonamido-macrocycles and the salts thereof, methods of preparing sulfonamido-macrocycles, a pharmaceutical composition containing said sulfonamido-macrocycles, use of said compounds as a medicament, and a method for treating diseases with said compounds, alt in accordance with the description, and as defined in the claims, of the present Application.

The application relates to a compound of the general Formula I: 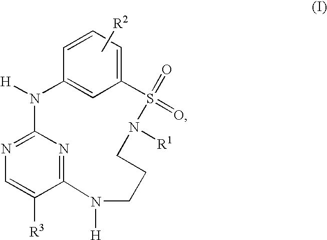
wherein
R¹ is hydrogen and —C₁-C₁₀-alkyl;
R² is selected from the group comprising, preferably consisting of, hydrogen, halogen, cyano, —NR⁴COR⁵, —NR⁴S(O)₂R⁵, NR⁴CONR⁵R⁶, —NR⁴COOR⁵, —COR⁴, —S(O)₂R⁴, —S(O)₂NR⁴R⁵ and CONR⁴R⁵;
R³ is selected from the group comprising, preferably consisting of, —C₁-C₆-alkyl, —C₂-C₆-alkenyl, —C₂-C₆-alkynyl, —C₃-C₈-cycloalkyl, C₆-C₁₁-aryl and —C₅-C₁₀-heteroaryl, wherein said residues are unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, —C₂-C₆-alkenyl, —C₂-C₆-alkynyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃ and wherein one or more C atoms of the C-backbone of —C₃-C₈-cycloalkyl being optionally singly or multiply replaced independently from each other by nitrogen atoms, oxygen atoms, sulfur atoms and/or C═O residues; or:
   optionally substituted phenyl-(CH₂)ₚ—COR⁴ or optionally substituted phenyl-(CH₂)ₚ—CONR⁴R⁵,
   wherein p is an integer of 1 to 4, preferably 1 to 3, more preferably 1 or 2;
R⁴, R⁵,
and R⁶ are the same or different and are independently from each other selected from the group comprising, preferably consisting of, hydrogen and residues being selected from the group comprising, preferably consisting of, —C₁-C₁₀-alkyl, —C₁-C₆-alkoxy, —C₂-C₆-alkenyl, —C₂-C₆-alkynyl, —C₃-C₈-cycloalkyl, —C₆-C₁₁-aryl and —C₅-C₁₀-heteroaryl, wherein said residues being unsubstituted or singly or multiply substituted independently from each other with hydroxy, halogen, —C₁-C₆-alkoxy, —C₁-C₆-alkylthio, amino, cyano, —C₁-C₆-alkyl, —C₃-C₁₀-cycloalkyl, —C₁-C₆-hydroxyalkyl, —C₂-C₆-alkenyl, —C₂-C₆-alkynyl, —C₁-C₆-alkoxy-C₁-C₆-alkyl, —NH—C₁-C₆-alkyl, —N(C₁-C₆-alkyl)₂, —S(O)(C₁-C₆-alkyl), —S(O)₂(C₁-C₆-alkyl), —C₁-C₆-alkanoyl, —CONR⁷R⁸, COR⁷, carboxy, —C₆-C₁₁-aryl, —C₅-C₁₀-heteroaryl or —NR⁷R⁸; wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃ and wherein one or more C atoms of the C-backbone of —C₃-C₈-cycloalkyl being optionally singly or multiply replaced independently from each other by nitrogen atoms, oxygen atoms, sulfur atoms and/or C═O— residues;
R⁷ and R⁸ are the same or different and are independently from each other selected from the group comprising, preferably consisting of, C₁-C₆-alkyl or phenyl, optionally substituted with halogen, hydroxy, —C₁-C₆-alkyl, —C₁-C₆-alkoxy, phenyl, —CF₃ and —OCF₃; and
   solvates, hydrates, N-oxides, isomers, diastereomers, enantiomers and salts thereof.

As used herein, the terms as mentioned hereinbelow and in the claims have preferably the following meanings:

As used herein, the term “alkyl” is to be understood as preferably meaning branched and unbranched alkyl, meaning e.g. methyl, ethyl, n-propyl, iso-propyl, n-butyl, iso-butyl, tert-butyl, sec-butyl, pentyl, iso-pentyl, hexyl, heptyl, octyl, nonyl and decyl and the isomers thereof.

As used herein, the term “alkoxy” is to be understood as preferably meaning branched and unbranched alkoxy, meaning e.g. methoxy, ethoxy, propyloxy, iso-propyloxy, butyloxy, iso-butyloxy, tert-butyloxy, sec-butyloxy, pentyloxy, iso-pentyloxy, hexyloxy, heptyloxy, octyloxy, nonyloxy, decyloxy, undecyloxy and dodecyloxy and the isomers thereof.

As used herein, the term “cycloalkyl” is to be understood as preferably meaning cycloalkyl, e.g. cyclopropyl, cyclobutyl, cyclopentyl, cyclohexyl and cycloheptyl. Cycloalkyl moieties which are singly or multiply interrupted by nitrogen atoms, oxygen atoms and/or sulfur atoms refer e.g. to oxiranyl, oxetanyl, aziridinyl, azetidinyl, tetrahydrofuranyl, pyrrolidinyl, morpholinyl, dithianyl, thiomorpholinyl, piperazinyl, trithianyl and chinuclidinyl. Cycloalkyl moieties, wherein the C-backbone contains one or more double bonds in the C-backbone refer e.g. to cycloalkenyl, such as cyclopropenyl, cyclobutenyl, cyclopentenyl, cyclohexenyl and cycloheptenyl, wherein the linkage can be provided to the double or single bond.

As used herein, the term “halogen” is to be understood as preferably meaning fluorine, chlorine, bromine or iodine.

As used herein, the term “alkenyl” is to be understood as preferably meaning branched and unbranched alkenyl, e.g. vinyl, propen-1-yl, propen-2-yl, but-1-en-1-yl, but-1-en-2-yl, but-2-en-1-yl, but-2-en-2-yl, but-1-en-3-yl, 2-methyl-prop-2-en-1-yl and 2-methyl-prop-1-en-1-yl.

As used herein, the term “alkynyl” is to be understood as preferably meaning branched and unbranched alkynyl, e.g. ethynyl, prop-1-yl-1-yl, but-1-yl-1-yl, but-2-yl-1-yl and but-3-yl-1-yl.

As used herein, the term aryl” is defined in each case as having 3-12 carbon atoms, preferably 6-12 carbon atoms, such as, for example, cyclopropenyl, cyclopentadienyl, phenyl, tropyl, cyclooctadienyl, indenyl, naphthyl, azulenyl, biphenyl, fluorenyl, anthracenyl etc, phenyl being preferred.

As used herein, the term “arylene” is understood as meaning cyclic or polycyclic aromatic groups, e.g. phenylene, naphthylene and biphenylene. More particularly, the term “phenylene” is understood as meaning ortho-, meta-, or para-phenylene. Preferably, this is meta-phenylene. As used herein, the term “heteroaryl” is understood as meaning an aromatic ring system which comprises 3-16 ring atoms, preferably 5 or 6 or 9 or 10 atoms, and which contains at least one heteroatom which may be identical or different, said heteroatom being such as oxygen, nitrogen or sulfur, and can be monocyclic, bicyclic, or tricyclic, and in addition in each case can be benzocondensed. Preferably, heteroaryl is selected from thienyl, furanyl, pyrrotyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, e.g., benzofuranyl, benzothienyl, benzoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, e.g., quinolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

As used herein, the term “heteroarylene” is understood as preferably meaning cyclic or polycyclic aromatic groups, e.g. to five-membered heteroaromatic groups, such as thiophenylene, furanylene, pyrrolylene, oxazolylene, thiazolylene, imidazolylene, pyrazolylene, triazolylene, thia-4H-pyrazolylene and benzo-derivates thereof, such as indolydene for example, or six-membered heteroaromatic groups, such as pyridinylene, pyrimidinylene, triazinylene and benzo-derivates thereof, e.g. quinolinylene, isoquinolinylene.

As used herein, the term “C₁-C₁₀”, as used throughout this text e.g. in the context of the definition of “C₁-C₁₀-alkyl”, is to be understood as meaning an alkyl group having a finite number of carbon atoms of 1 to 10, i.e. 1, 2, 3, 4, 5, 6, 7, 8, 9, or 10 carbon atoms. It is to be understood further that said term “C₁-C₁₀” is to be interpreted as any sub-range comprised therein, e.g. C₁-C₁₀, C₂-C₉, C₃-C₈, C₄-C₇, C₅-C₆, C₁-C₂, C₁-C₃, C₁-C₄, C₁-C₅, C₁-C₆, C₁-C₇, C₁-C₈, C₁-C₉; preferably C₁-C₂, C₁-C₃, C₁-C₄, C₁-C₅, C₁-C₆; more preferably C₁-C₃.

Similarly, as used herein, the term “C₁-C₆”, as used throughout this text e.g. in the context of the definition of “C₁-C₆-alkyl”, “C₁-C₆-alkoxy”, “C₁-C₆-alkylthio”, “C₁-C₆-hydroxyalkyl”, “C₁-C₆-alkoxy-C₁-C₆-alkyl”, “—NH—C₁-C₆-alkyl”, “—N(C₁-C₆-alkyl)₂”, “—S(O)₂(C₁-C₆-alkyl)” and “—C₁-C₆-alkanoyl”, is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅C₁-C₆; preferably C₁-C₂, C₁-C₃, C₁-C₄, C₁-C₅, C₁-C₆; more preferably C₁-C₃.

Similarly, as used herein, the term “C₂-C₆”, as used throughout this text e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; preferably C₂-C₃

Further, as used herein, the term “C₃-C₈”, as used throughout this text e.g. in the context of the definitions of “C₃-C₈-cycloalkyl”, is to be understood as meaning a cycloalkyl group having a finite number of carbon atoms of 3 to 8, i.e. 3, 4, 5, 6, 7 or 8 carbon atoms, preferably 5 or 6 carbon atoms. It is to be understood further that said term “C₃-C₈” is to be interpreted as any sub-range comprised therein, e.g. C₃-C₈, C₄-C₇, C₅-C₆, C₃-C₄, C₃-C₅, C₃-C₆, C₃-C₇; preferably C₅-C₆.

Even further, as used herein, the term “C₆-C₁₁”, as used throughout this text e.g. in the context of the definitions of “C₆-C₁₁-aryl”, is to be understood as meaning an aryl group having a finite number of carbon atoms of 6 to 11, i.e. 6, 7, 8, 9, 10, or 11 carbon atoms, preferably 5, 6 or 10 carbon atoms. It is to be understood further that said term “C₆-C₁₁” is to be interpreted as any sub-range comprised therein, e.g. C₆-C₁₁, C₇-C₁₀, C₈-C₉, C₉-C₁₀; preferably C₅-C₆ or C₉-C₁₀. Similarly, as used herein, the term “C₅-C₁₀”, as used throughout this text e.g. in the context of the definitions of “C₅-C₁₀-heteroaryl”, is to be understood as meaning a heteroaryl group having a finite number of carbon atoms of 5 to 10, i.e. 5, 6, 7, 8, 9, or 10 carbon atoms, preferably 5, 6 or 10 carbon atoms, of which at least one carbon atom is replaced by a heteroatom, said heteroatom being as defined supra. It is to be understood further that said term “C₅-C₁₀” is to be interpreted as any sub-range comprised therein, e.g. C₅-C₁₀, C₆-C₉, C₇-C₈; preferably C₅-C₆ or C₉-C₁₀.

The compound according to Formula I (sulfonamido-macrocycles) can exist as N-oxides which are defined in that at least one nitrogen of the compounds of the general Formula I may be oxidized.

The compound according to Formula I (sulfonamido-macrocycles) can exist as solvates, in particular as hydrate, wherein the compound according to Formula I may contain polar solvents, in particular water, as structural element of the crystal lattice of the compounds. The amount of polar solvents, in particular water, may exist in a stoichiometric or unstoichiometric ratio. In case of stoichiometric solvates, e.g. hydrate, are possible hemi-, (semi-), mono-, sesqui-, di-, tri-, tetra-, penta- etc. solvates or hydrates, respectively.

As used herein, the term “isomers” refers to chemical compounds with the same number and types of atoms as another chemical species. There are two main classes of isomers, constitutional isomers and stereoisomers.

As used herein, the term “constitutional isomers” refers to chemical compounds with the same number and types of atoms, but they are connected in differing sequences. There are functional isomers, structural isomers, tautomers or valence isomers.

In stereoisomers, the atoms are connected sequentially in the same way, such that condensed formulae for two isomeric molecules are identical. The isomers differ, however, in the way the atoms are arranged in space. There are two major sub-classes of stereoisomers; conformational isomers, which interconvert through rotations around single bonds, and configurational isomers, which are not readily interconvertable.

Configurational isomers are, in turn, comprised of enantiomers and diastereomers. Enantiomers are stereoisomers which are related to each other as mirror images. Enantiomers can contain any number of stereogenic centers, as long as each center is the exact mirror image of the corresponding center in the other molecule. If one or more of these centers differs in configuration, the two molecules are no Longer mirror images. Stereoisomers which are not enantiomers are called diastereomers. Diastereomers which still have a different constitution, are another sub-class of diastereomers, the best known of which are simple cis-trans isomers.

In order to limit different types of isomers from each other reference is made to IUPAC Rules Section E (Pure Appl Chem 45, 11-30, 1976).

As used herein, the term “in vivo hydrolysable ester” is understood as meaning an in vivo hydrolysable ester of a compound of formula (I) containing a carboxy or hydroxy group is, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl and C1-6alkoxycarbonyloxyethyl esters, e.g. 1-methoxycarbonyloxyethyl, and may be formed at any carboxy group in the compounds of this invention. An in vivo hydrolysable ester of a compound of formula (I) containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

The compound according to Formula I (sulfonamido-macrocycles) can exist in free form or in a salt form. A suitably pharmaceutically acceptable salt of the sulfonamido-macrocycles of the invention is, for example, an acid-addition salt of a sulfonamido-macrocycle of the invention which is sufficiently basic, for example, an acid-addition salt with, for example, an inorganic or organic acid, for example hydrochloric, hydrobromic, sulfuric, phosphoric, trifluoroacetic, citric or maleic acid. In addition a suitably pharmaceutically acceptable salt of a sulfonamido-macrocycle of the invention which is sufficiently acidic is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, 1,6-hexadiamine, ethanolamine, glucosamine, sarkosine, serinol, tris-hydroxy-methyl-aminomethane, aminopropandiol, sovak-base, 1-amino-2,3,4-butantriol.

Compounds of Formula I according to the present invention are particular preferred, wherein R³ is —C₁-C₆-alkyl being unsubstituted or singly or multiply substituted independently from each other with hydroxy, halogen, —C₁-C₆-alkoxy, —C₁-C₆-alkylthio, amino, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R^{wherein —C}₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

Further compounds are preferred wherein R³ is —C₂-C₆-alkenyl, as defined supra, being unsubstituted or singly or multiply substituted independently from each other with hydroxy, halogen, —C₁-C₆-alkoxy, amino, —C₁-C₆-alkyl, —C₁-C₆-hydroxyalkyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

Further compounds are preferred wherein R³ is —C₂-C₆-alkynyl, as defined supra, being unsubstituted or singly or multiply substituted independently from each other with hydroxy, halogen, —C₁-C₆-alkoxy, amino, —C₁-C₆-alkyl, —C₁-C₆-hydroxyalkyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

Further compounds are preferred wherein R³ is —C₅-C₇-cycloalkyl, as defined supra, being unsubstituted or singly or multiply substituted independently from each other with hydroxy, halogen, —C₁-C₆-alkyl, —S(O)₂(C₁-C₆-alkyl), —C₁-C₆-alkanoyl, —CONR⁴R⁵ and/or —COR⁴, wherein one or more C atoms of the C-backbone of —C₅-C₇-cycloalkyl being optionally replaced independently from each other by one or two nitrogen atoms, wherein in this case those compounds are especially preferred, wherein R³ is piperazine or piperidine, wherein piperazine or piperidine being unsubstituted or singly or multiply substituted independently from each other with —C₁-C₆-alkyl, —S(O)₂(C₁-C₆-alkyl), —C₁-C₆-alkanoyl, —CONR⁴R⁵ and/or —COR⁴.

Further compounds are preferred wherein R³ is:

either:

—C₆-C₁₁-aryl, as defined supra, preferably phenyl, being unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃;

or:

optionally substituted phenyl-(CH₂)ₚ—COR⁴ or optionally substituted phenyl-(CH₂)ₚ—CONR⁴R⁵, wherein p is an integer of 1 to 3, more preferably 1 or 2;

More advantageously, compounds are preferred wherein:

R³ is either:

phenyl unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃, —C₁-C₆-alkyl, —C₁-C₆-alkoxy, —CF₃ or —OCF₃;

or:

optionally substituted phenyl-(CH₂)ₚ—COR⁴ or optionally substituted phenyl-(CH₂)ₚ—CONR⁴R⁵,

wherein p is an integer of 1 to 3, more preferably 1 or 2;

Even more advantageously, compounds are preferred wherein

R³ is either:

phenyl unsubstituted or singly or multiply substituted independently from each other with halogen, —C₁-C₆-alkoxy, —C₁-C₆-alkyl, —C₁-C₆-hydroxyalkyl, C₁-C₆-alkoxy-C₁-C₆-alkyl, —NH—C₁-C₆-alkyl, —N(C₁-C₆-alkyl)₂, —C₁-C₆-alkanoyl, —COR⁴, and/or —NR⁴R⁵;

or:

optionally substituted phenyl-(CH₂)ₚ—COR⁴ or optionally substituted phenyl-(CH₂)ₚ—CONR⁴R⁵,

wherein p is an integer of 1 or 2.

Particularly advantageously, compounds are preferred wherein:

R³ is phenyl singly or multiply substituted independently from each other with C₁-C₆-alkoxy, —C₁-C₆-alkyl.

Even more advantageously, compounds are preferred wherein R³ is phenyl.

The compounds of the present invention can be used in treating diseases of dysregulated vascular growth or diseases which are accompanied by dysregulated vascular growth. Especially, the compounds effectively interfere with angiopoietin and therefore influence Tie2 signalling. Surprisingly, the compounds block Tie2 signalling, wherein Tie2 kinase activity is blocked with showing no or very low cell toxicity for cells other than endothelial cells at low concentrations, which is an important advantage over prior art substances. This effect can therefore allow prolonged treatment of patients with the compounds offering good tolerability and high anti-angiogenic efficacy, where persistent angiogenesis plays a pathologic role.

Therefore the compounds of the present invention can be applied for the treatment of diseases accompanied by neoangiogenesis. This holds principally for all solid tumours, e.g. breast, colon, renal, lung and/or brain tumours and can be extended to a broad range of diseases, where pathologic angiogenesis is persistent. This applies for diseases with inflammatory association, diseases associated with oedema of various forms and diseases associated with stromal proliferation and pathologic stromal reactions broadly. Particularly suited is the treatment for gynaecological diseases where inhibition of angiogenic, inflammatory and stromal processes with pathologic character can be inhibited. At the same time the toxic side effects on normal proliferating tissue are tow. The treatment is therefore an addition to the existing armament to treat diseases associated with neoangiogenesis.

The compounds of the present invention can be used in particular in therapy and prevention of tumour growth and metastases especially in solid tumours of all indications and stages with or without pre-treatment if the tumour growth is accompanied with persistent angiogenesis. However it is not restricted to tumour therapy but is also of great value for the treatment of other diseases with dysregulated vascular growth. This includes retinopathy and other angiogenesis dependent diseases of the eye (e.g. cornea transplant rejection, age-related macular degeneration), rheumatoid arthritis, and other inflammatory diseases associated with angiogenesis such as psoriasis, delayed type hypersensitivity, contact dermatitis, asthma, multiple sclerosis, restenosis, pulmonary hypertension, stroke and inflammatory diseases of the bowel, such as Crohn's disease. It includes coronary and peripheral artery disease. It can be applied for disease states such as ascites, oedema, such as brain tumour associated oedema, high altitude trauma, hypoxia induced cerebral oedema, pulmonary oedema and macular oedema or oedema following burns and trauma. Furthermore, it is useful for chronic lung disease, adult respiratory distress syndrome. Also for bone resorption and for benign proliferating diseases such as myoma, benign prostate hyperplasia and wound healing for the reduction of scar formation. It is therapeutically valuable for the treatment of diseases, where deposition of fibrin or extracellular matrix is an issue and stroma proliferation is accelerated (e.g. fibrosis, cirrhosis, carpal tunnel syndrome etc). In addition, it can be used for the reduction of scar formation during regeneration of damaged nerves, permitting the reconnection of axons. Further uses are endometriosis, pre-eclampsia, postmenopausal bleeding and ovarian hyperstimulation.

A second aspect of the invention is a pharmaceutical composition which contains a compound of Formula I or pharmaceutically acceptable salts thereof, isomers or mixtures of isomers thereof, in admixture with one or more suitable excipients. This composition is particularly suited for the treatment of diseases of dysregulated vascular growth or of diseases which are accompanied with dysregulated vascular growth as explained above.

In order that the compounds of the present invention be used as pharmaceutical products, the compounds or mixtures thereof may be provided in a pharmaceutical composition, which, as well as the compounds of the present invention for enteral, oral or parenteral application, contain suitably pharmaceutically acceptable organic or inorganic inert base material, e.g. purified water, gelatin, gum Arabic, lactate, starch, magnesium stearate, talc, vegetable oils, polyalkylenglycol, etc.

The pharmaceutical composition may be provided in a solid form, e.g. as tablets, dragees, suppositories, capsules or in liquid form, e.g. as a solution, suspension or emulsion. The pharmaceutical composition may additionally contain auxiliary substances, e.g. preservatives, stabilisers, wetting agents or emulsifiers, salts for adjusting the osmotic pressure or buffers.

For parenteral applications (including intravenous, subcutaneous, intramuscular, intravascular or infusion) sterile injection solutions or suspensions are preferred, especially aqueous solutions of the compounds in polyhydroxyethoxy containing castor oil.

The pharmaceutical compositions of the present invention may further contain surface active agents, e.g. salts of gallenic acid, phospholipids of animal or vegetable origin, mixtures thereof and liposomes and parts thereof.

For oral application tablets, dragees or capsules with talc and/or hydrocarbon-containing carriers and binders, e.g. lactose, maize and potato starch, are preferred. Further application in liquid form is possible, for example as juice, which contains sweetener if necessary.

The dosage will necessarily be varied depending upon the route of administration, age, weight of the patient, the kind and severity of the illness being treated and similar factors. The daily dose is in the range of 0.5-1,500 mg. A dose can be administered as unit dose or in part thereof and distributed over the day. Accordingly the optimum dosage may be determined by the practitioner who is treating any particular patient.

Another aspect of the present invention is a method which may be used for preparing the compounds according to the present invention.

The following table lists the abbreviations used in this paragraph, and in the Examples section. NMR peak forms are stated as they appear in the spectra, possible higher order effects have not been considered.

| Abbreviation | Meaning |
|---|---|
| Ac | acetyl |
| Boc | tert-butyloxycarbonyl |
| br | broad |
| c- | cyclo- |
| CI | chemical ionisation |
| d | doublet |
| dd | doublet of doublet |
| DCM | dichloromethane |
| DIPEA | N,N-diisopropylethyl amine |
| DMF | N,N-dimethylformamide |
| DMSO | dimethyl sulfoxide |
| eq. | equivalent |
| ESI | electrospray ionisation |
| GP | general procedure |
| m | multiplet |
| mc | centred multiplet |
| MS | mass spectrometry |
| NMR | nuclear magnetic resonance spectroscopy: |
| | chemical shifts (δ) are given in ppm. |
| POPd | dihydrogen dichlorobis(di-tert-butyl phosphinito- |
| | □P)palladate(2); CombiPhos Catalysts, Inc. |
| q | quartet |
| s | singlet |
| t | triplet |
| TEA | triethylamine |
| TFA | trifluoroacetic acid |
| THF | tetrahydrofuran |

The following schemes and general procedures illustrate general synthetic routes to the compounds of general Formula I of the invention and are not intended to be limiting. Specific examples are described in the subsequent paragraph. Thus, the compounds of the invention can be prepared starting from halogenated macrocycles (A) by metal-catalysed coupling reactions such as Suzuki, Heck, or Sonogashira couplings, particularly a coupling reaction catalysed by a transition metal, e.g. Cu, Pd, or by amination methods well known to the person skilled in the art.

One first, general reaction scheme is outlined hereinbelow

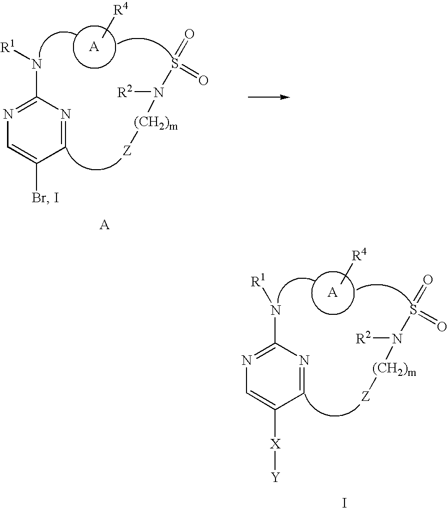

Scheme 1: Coupling, e.g. Suzuki or Ullmann coupling of macrocycles A, wherein A in macrocycles A is preferably phenylene, Z is —NR³ and m=3, wherein in Formula I X is a bond and Y is e.g. -phenylene-D-NH—COR⁵, -phenylene-D-NH—CONR⁵R⁶, -phenylene-D-NH—S(O)₂R⁵, -phenylene-D-O—(CH₂)ₚ—COR⁵, -oxy-C₆-C₁₁-aryl, or -oxy-C₅-C₁₀-heteroaryl with the meaning above.

A second, particular reaction scheme is outlined hereinbelow:

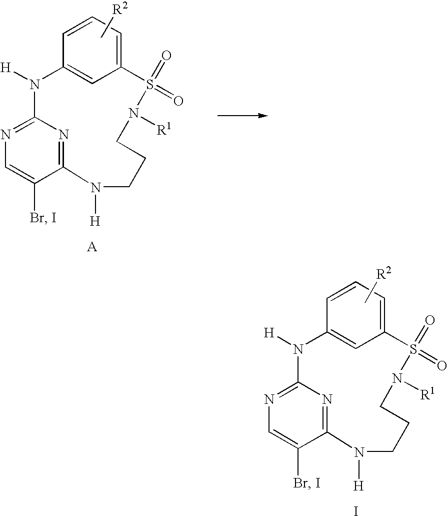

Scheme 2: Amination of A with cyclic aliphatic amines, e.g. N-Boc-piperazine can be used. The Amination of macrocycle A gives compounds of Formula I in which R³ is preferably unsubstituted or substituted —C₃-C₁₀-cycloalkyl containing at least one hetero atom, e.g. nitrogen atom, which replaces the halide in the macrocycle A.

The synthesis of the halogenated macrocycle A is described in WO 2004/026881 A, and is herein exemplified as Preparation Example A, particularly with respect to the brominated macrocycle A, and as Preparation Example B, with respect to the iodinated macrocycle A.

### Preparation Example A

### Production of 1⁵-Bromo-4-thia-2,5,9-triaza-1(2,4)-pyrimidina-3(1,3)-benzenacyclononaphane-4,4-dioxide

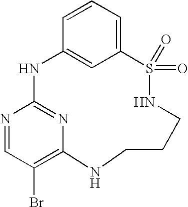 Method A

A solution of 200 mg (0.48 mmol) of 3-amino-N-[3-(5-bromo-2-chloro-pyrimidin-4-ylamino)-propyl]-benzenesulfonamide in acetonitrite/water/2-butanol (9.0 ml/1.0 ml/0.3 ml) is added via a syringe pump within 2.5 hours to a refluxing mixture of acetonitrite/water/4 molar solution of hydrochloric acid in dioxane (45 ml/5 ml/0.6 ml). After another 3 hours under reflux, the oil bath is turned off, and the reaction solution is stirred overnight at room temperature. The precipitate that is formed is filtered off, washed with water and then dried in a vacuum. 112 mg (0.31 mmol) of the product is obtained. The filtrate is concentrated by evaporation in a rotary evaporator. The precipitate that is formed is washed with water and filtered off. After drying, another 45 mg (0.12 mmol) of the product is obtained. The total yield of product is thus 157 mg (0.41 mmol, corresponding to 85% of theory).

Method B

A solution of 450 mg (1.00 mmol) of N-[3-(5-bromo-2-chloro-pyrimidin-4-ylamino)-propyl]-3-nitro-benzenesulfonamide in 9.5 ml of ethanol is mixed with 960 mg of tin(II) chloride and stirred for 30 minutes at 70° C. After cooling, the reaction mixture is carefully added to ice water and made basic with 1N NaOH solution. It is extracted with ethyl acetate (3×). The combined organic phases are dried (Na₂SO₄), filtered and concentrated by evaporation. The remaining residue is purified by chromatography (ethyl acetate/hexane 4:1). 72 mg of the crude product is obtained. It is mixed with 1N HCL and extracted with ethyl acetate. A colorless solid precipitates from the aqueous phase. The solid is filtered off and dried. 20 mg (0.05 mmol, corresponding to 5% of theory) of the product is obtained.

¹H-NMR (DMSO): 10.45 (s, 1H), 9.07 (s, 1H), 8.35 (br, 1H), 8.18 (s, 1H), 7.78 (t, 1H), 7.45 (m, 2H), 7.32 (m, 1H), 3.44 (m, 2H), 3.28 (m, 2H), 1.82 (m, 2H).

MS: 384 (ES).

Production of the Intermediate Product

### Production of 3-Amino-N-[3-(5-bromo-2-chloro-pyrimidin-4-ylamino)-propyl]-benzenesulfonamide

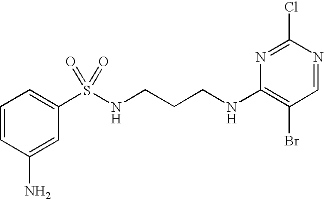

A solution of 1.35 g (2.99 mmol) of N-[3-(5-bromo-2-chloro-pyrimidin-4-ylamino)-propyl]-3-nitro-benzenesulfonamide in 100 ml of tetrahydrofuran is mixed under argon at room temperature with 15 ml of a 15% solution of Ti(III)Cl₃ in about 10% hydrochloric acid. After 17 hours, the reaction solution is mixed again with 1 ml of the Ti(III)Cl₃ solution and stirred for another 3 hours. The batch is made basic with 1N NaOH solution and then filtered. The filter cake is rewashed 2× with 100 ml of ethyl acetate/MeOH (30 ml/20 ml) in each case. The filtrate is concentrated by evaporation in a rotary evaporator and then extracted with ethyl acetate (2×). The combined organic phases are washed with NaCl solution, dried (Na₂SO₄), filtered and concentrated by evaporation. The remaining residue is purified by chromatography (dichloromethane/MeOH 95:5, Flashmaster II). 624 mg (1.48 mmol, corresponding to 49% of theory) of the product is obtained.

¹H-NMR (DMSO): 8.21 (s, 1H), 7.63 (t, 1H), 7.38 (t, 1H), 7.13 (t, 1H), 6.97 (m, 1H), 6.83 (m, 1H), 6.71 (m, 1H), 5.53 (s, 2H), 3.30 (m, 2H), 2.75 (m, 2H), 1.65 (m, 2H).

### Preparation Example B

### Production of 1⁵-Iodo-4-thia-2,5,9-triaza-1(2,4)-pyrimidina-3(1,3)-benzenacyclononaphane-4,4-dioxide

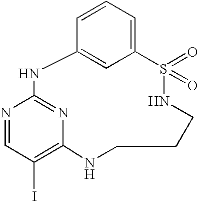

A solution of 2.34 g (5.00 mmol) of 3-amino-N-[3-(5-iodo-2-chloro-pyrimidin-4-ylamino)-propyl]-benzenesulfonamide in acetonitrile/water/2-butanol (94 mL/10.4 mL/3.1 mL) is added via a syringe pump within 3 hours to a refluxing mixture of acetonitrile/water/4 molar solution of hydrochloric acid in dioxane (470 mL/52 mL/6.2 mL). After another 3 hours under reflux, the heating of the respective oil bath is switched off, and the reaction solution is stirred overnight at room temperature. The precipitate that is formed is filtered off, washed with acetonitrile and then dried in vacuo to give 1.71 g (79% yield) of the desired product.

¹H-NMR (DMSO, 300 MHz): 10.81 (s, 1H), 9.02 (s, 1H), 8.30-8.38 (m, 1H), 8.27 (s, 1H), 7.82 (t, 1H), 7.43-7.56 (m, 2H), 7.29-7.40 (m, 1H), 3.38-3.52 (m, 2H), 3.21-3.36 (m, 2H), 1.72-1.90 (m, 2H).

ESI-MS: [M+H⁺]=432.

Production of the Intermediate Product

### Production of 3-Amino-N-[3-(5-iodo-2-chloro-pyrimidin-4-ylamino)-propyl]-benzenesulfonamide

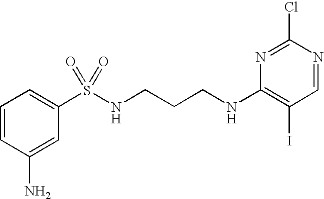

A solution of 9.95 g (20.0 mmol) of N-[3-(5-iodo-2-chloro-pyrimidin-4-ylamino)-propyl]-3-nitro-benzenesulfonamide in 660 mL of tetrahydrofuran is mixed under argon at room temperature with 100 mL of a 15% solution of Ti(III)Cl₃ in about 10% hydrochloric acid. After 2 hours, the reaction solution is mixed again with 7 mL of the Ti(III)Cl₃ solution and is additionally stirred for one hour. The mixture is made basic (pH 14) by addition of 1N NaOH solution and then filtered over Celite. The filtrate is extracted with ethyl acetate (3×400 mL), the combined organic layers are then washed with brine (200 mL), and concentrated in vacuo. The filter cake is rewashed 4× with 500 ml of ethyl acetate/MeOH (3:2), followed by evaporation of the resulting washing fractions. The resulting residues are combined and purified by column chromatography over silica (dichloromethane/ethyl acetate) to give 5.42 g (58% yield) of the target compound.

¹H-NMR (DMSO, 300 MHz): 8.31 (s, 1H), 7.39 (t, 1H), 7.27 (t, 1H), 7.16 (t, 1H), 6.95-7.01 (m, 1H), 6.82-6.88 (m, 1H), 6.68-6.76 (m, 1H), 5.53 (s, 2H), 3.27-3.39 (m, 2H), 2.68-2.82 (m, 2H), 1.64 (mc, 2H).

ESI-MS: [M+H⁺]=468 (³⁵Cl signal; ³⁷Cl isotope also well detected).

Examples 1 to 43 were prepared employing the following general procedure for Suzuki couplings:

General Procedure 1 (GP1): Suzuki Coupling

(Typical Scale: 0.25 mmol)

A solution of the respective macrocyclic halide in DMF (8 mL per mmol halide) was treated with the respective organoboron compound (1.25 eq.), K₂CO₃ (2.5 eq., either as a solid or as 2 M aqueous solution), and POPd (2.5-5 mol-%) at room temperature. The stirred resulting mixture was placed into an oil bath preheated to 100° C. The reaction progress was monitored by TLC, and in case of incomplete turnover of the macrocyclic halide after 2 h additional portions of POPd and the organoboron compound were added followed by additional stirring at 100° C. After cooling to room temperature, water was added and the resulting suspension was stirred for 30 min. The crude product was isolated by vacuum filtration, dried in vacuo, and purified by column chromatography, followed optionally by trituration with methanol and/or preparative HPLC (e.g. YMC Pro C18RS 5μ, 150×20 mm, 0.2% NH₃ in water/acetonitrile) to yield the analylically pure products. Alternatively, after full conversion the reaction mixture was diluted with ethyl acetate, quenched with water. Layers were separated, the organic layer was extracted with ethyl acetate twice and the combined organic layers dried and concentrated in vacuo followed by the above mentioned further purification steps.

In all except the explicitly stated examples boronic acids were used as organoboron compounds for the above described general Suzuki procedure.

The preparation of commercially not available organoboron compounds used as substrates for Suzuki couplings is described in the following sections.

### Intermediate I

### Preparation of 1-Phenyl-3-[4-(4,4,5,5-tetramethyl-1,3,2-dioxaborolan-2-yl)-phenyl]-propan-1-one (Scheme A)

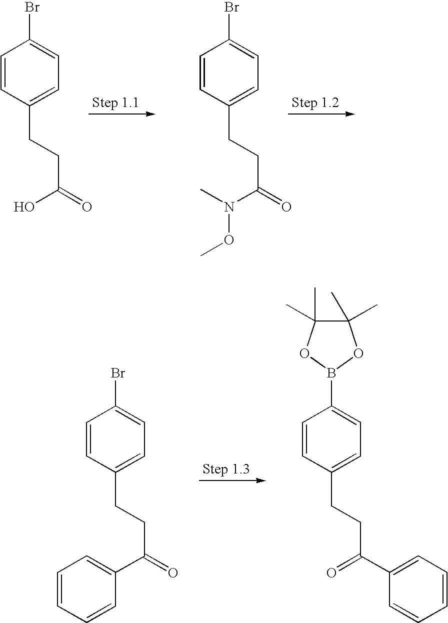

### Scheme A

### Preparation of 3-(4-Bromophenyl)-N-methoxy-N-methyl-propionamide (Step 1.1)

4.0 g of 3-(4-bromophenyl)propionic acid (17.5 mmol) were dissolved in 45 mL THF (0.4 M) and treated subsequently with 5.75 mL N-methylmorpholine (52.4 mmol, 3.0 eq.) and 3.7 g 2-chloro-4,6-dimethoxy[1,3,5]triazine (CDMT, 20.95 mmol, 1.2 eq.). The resulting mixture was stirred for 1 h at room temperature upon which 1.87 g HNMe(OMe).HCL (19.2 mmol, 1.1 eq.) were added and stirring was continued for 16 h. The reaction was quenched with water and extracted with ethyl acetate, the combined organic layers were washed with saturated aq. Na₂CO₃-solution, with 1 N HCL, subsequently dried and concentrated in vacuo to yield 4.38 g of 3-(4-Bromophenyl)-N-methoxy-N-methyl-propionamide (92%) after flash column chromatography.

¹H-NMR (DMSO, 300 MHz): 7.64 (d, 2H); 7.19 (d, 2H); 3.58 (s, 3H); 3.04 (s, 3H); 2.74 (t, 2H); 2.64 (t, 2H).

### 3-(4-Bromophenyl)-1-phenyl-propan-1-one (Step 1.2)

700 mg of 3-(4-Bromophenyl)-N-methoxy-N-methyl-propionamide (2.57 mmol) were dissolved in 13 mL dry THF (0.2 M), cooled to 0° C. and treated dropwise with 3.1 mL phenylmagnesium bromide solution (1.0 M in THF, 3.1 mmol., 1.2 eq.). The reaction was stirred for 2 h at 0° C., quenched with 1N HCL, extracted with ethyl acetate, the combined organic extracts dried and concentrated in vacuo. Flash column chromatography yielded 509 mg (68%) of 3-(4-Bromo-phenyl)-1-phenyl-propan-1-one as a white solid.

¹H-NMR (CDCl₃, 300 MHz): 7.96 (d, 2H); 7.58 (m, 1H); 7.40-7.50 (m, 4H); 7.16 (d, 2H); 3.60 (t, 2H); 3.08 (t, 2H).

1-Phenyl-3-[4-(4,4,5,5-tetramethyl-1,3,2-dioxaborolan-2-yl)-phenyl]-propan-1-one (Step 1.3)

100 mg of 3-(4-Bromophenyl)-1-phenyl-propan-1-one (0.35 mmol), 133 mg bis(pinacolato)diboron (0.525 mmol, 1.5 eq.), 103 mg KOAc (1.05 mmol, 3.0 eq.) and 29 mg PdCl₂(dppf).CH₂Cl₂ complex (0.035 mmol, 10 mol %) were weighed into a flame-dried Schlenk flask and set under an atmosphere of argon. 1.8 mL DMSO were added and the resulting suspension was stirred at 80° C. for 5 h. The reaction was diluted with ethyl acetate, quenched with water, filtered through Celite and extracted with ethyl acetate. The combined organic layers were washed with brine twice, dried and concentrated in vacuo. Flash column chromatography provided 89 mg (76%) of 1-Phenyl-3-[4-(4,4,5,5-tetramethyl-1,3,2-dioxaborolan-2-yl)-phenyl]-propan-1-one as a colourless solid.

¹H-NMR (CDCl₃, 300 MHz): 7.97 (d, 2H); 7.76 (d, 2H); 7.56 (t, 1H); 7.43 (t, 2H); 7.27 (d, 2H); 3.32 (t, 2H); 3.10 (t, 2H); 1.37 (s, 12H).

The following Intermediates 2 to 7 were prepared in analogous fashion to steps 1.1 to 1.3 by using appropriately functionalized Grignard reagents:

| Intermediate | | | |
|---|---|---|---|
| No | Structure | Name | Analytical data |
| 2 | 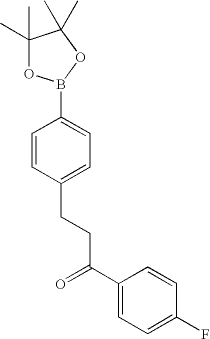 | 1-(4-Fluoro- phenyl)-3-[4- (4,4,5,5- tetramethyl-1,3,2- dioxaborolan-2- yl)-phenyl]- propan-1-one | ¹H-NMR (CDCl₃, 300 MHz): 7.97 (d, 2 H); 7.76 (d, 2 H); 7.26 (d, 2 H); 7.12 (d, 2 H); 3.26 (t, 2 H); 3.08 (t, 2 H); 1.34 (s, 12 H). |
| 3 | 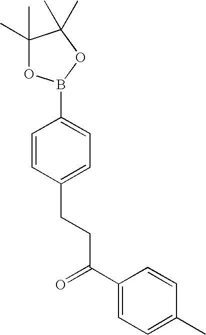 | 1-(4- Methylphenyl)-3- [4-(4,4,5,5- tetramethyl-1,3,2- dioxaborolan-2- yl)phenyl]propan- 1-one | ¹H-NMR (CDCl₃, 400 MHz): 7.86 (d, 2 H); 7.74 (d, 2 H); 7.21-7.28 (m, 4 H); 3.28 (t, 2 H); 3.08 (t, 2 H); 2.40 (s, 3 H); 1.34 (s, 12 H). |
| 4 | 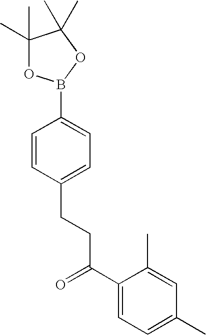 | 1-(2,4-Dimethyl- phenyl)-3-[4- (4,4,5,5- tetramethyl-1,3,2- dioxaborolan-2- yl)-phenyl]- propan-1-one | ¹H-NMR (CDCl₃, 400 MHz): 7.73 (d, 2 H); 7.57 (d, 1H); 7.24 (d, 2 H); 7.05 (s, 1 H); 7.04 (s, 1 H); 3.19 (q, 2 H); 3.04 (t, 2 H); 2.46 (s, 3 H); 2.34 (s, 3 H); 1.34 (s, 12 H). |
| 5 | 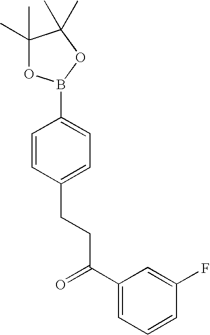 | 1-(3-Fluoro- phenyl)-3-[4- (4,4,5,5- tetramethyl-1,3,2- dioxaborolan-2- yl)-phenyl]- propan-1-one | ¹H-NMR (CDCl₃, 300 MHz): 7.74 (d, 2 H); 7.71 (br. s, 1 H); 7.64 (dd, 1 H); 7.43 (m, 1 H); 7.21-7.28 (m, 3 H); 3.28 (t, 2 H); 3.06 (t, 2 H); 1.36 (s, 12 H). |
| 6 | 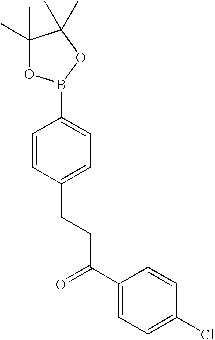 | 1-(4-Chloro- phenyl)-3-[4- (4,4,5,5- tetramethyl-1,3,2- dioxaborolan-2- yl)-phenyl]- propan-1-one | ¹H-NMR (CDCl₃, 400 MHz): 7.89 (d, 2 H); 7.75 (d, 2 H); 7.44 (d, 2 H); 7.26 (d, 2 H); 3.26 (t, 2 H); 3.08 (t, 2 H); 1.36 (s, 12 H). |
| 7 | 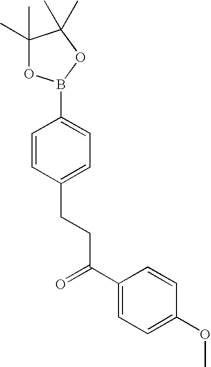 | 1-(4-Methoxy- phenyl)-3-[4- (4,4,5,5- tetramethyl-1,3,2- dioxaborolan-2- yl)-phenyl]- propan-1-one | ¹H-NMR (CDCl₃, 300 MHz): 7.94 (d, 2 H); 7.74 (d, 2 H); 7.27 (d, 2 H); 6.93 (d, 2 H); 3.86 (s, 3 H); 3.25 (t, 2 H); 3.08 (t, 2 H); 1.34 (s, 12 H). |

Examples 44 to 48 were prepared by by amide formation of Intermediates 8 and 9 with the respective amines following general procedure GP3. Intermediates 8 and 9 are derived from the respective carboxylic acid esters employing general procedure GP2.

General Procedure 2 (GP 2): Saponification of Alkyl Esters

To a suspension or solution of the respective ester in isopropanol (10 mL per mmol) was added 0.5 M aqueous sodium hydroxide (20 eq), and the resulting mixture was stirred for 6 h at 60° C. Additional sodium hydroxide was added on demand, and stirring was continued until the reaction was completed according to TLC. Diluted acetic acid (1.0 M) was added until pH 4 was maintained, and the precipitated acid was isolated by filtration, washed with water, and dried in vacuo, and then used without further purification.

General Procedure 3 (GP3): Amide Couplings with EDC/HOBt

To a solution of the respective carboxylic acid in DMAc (20 mL per mmol) were subsequently added HOBt (0.11 eq), EDC (1.0 eq), and TEA (2.0 eq) at room temperature under an atmosphere of nitrogen. The resulting mixture was stirred for 45 min, after which the respective amine was added as a 0.1M solution in DMAc. After overnight stirring, the mixture was concentrated in vacuo, diluted with water, and stirred at room temperature for 30 min. The precipitate was isolated by filtration and then dried. Purification of the crude product was performed by preparative HPLC (e.g. on a Gemini C18 5μ250×21.2 mm column with a water/acetonitrile gradient), or column chromatography, optionally followed by trituration with methanol.

### Intermediate 8

### Preparation of 4-(4,4-Dioxo-4-thia-2,5,9-triaza-1(2,4)-pyrimidina-3(1,3)-benzenacyclononaphan-1⁵-yl)benzenepropanoic acid

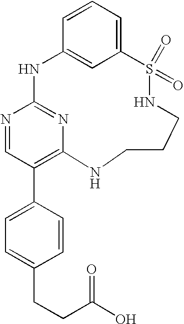

Intermediate 8 was prepared according to GP 2 from Methyl 4-(4,4-dioxo-4-thia-2,5,9-triaza-1(2,4)-pyrimidina-3(1,3)-benzenacyclononaphan-1⁵-yl)benzene-propanoate (Example 41) in quantitative yield.

¹H-NMR (CDCl₃, 400 MHz): 12.14 (s br, 1H); 9.53 (s, 1H); 9.42 (s, 1H); 7.70-7.76 (m, 2H); 7.34 (mc, 1H); 7.21-7.31 (m, 6H); 6.88 (t br, 1H); 3.21-3.42 (m, 4H); 2.83 (t, 2H); 2.53 (t, 2H); 1.76-1.86 (m, 2H). MS (ESI): [M−H]⁻=454.

### Intermediate 9

### Preparation of 4-(4,4-Dioxo-4-thia-2,5,9-triaza-1(2,4)-pyrimidina-3(1,3)-benzenacyclononaphan-1⁵-yl)benzeneacetic acid

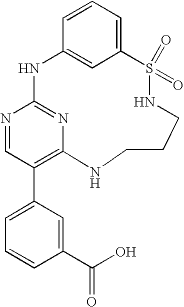

Intermediate 9 was prepared according to GP2 from Methyl 4-(4,4-dioxo-4-thia-2,5,9-triaza-1(2,4)-pyrimidina-3(1,3)-benzenacyclononaphan-1⁵-yl)benzene-acetate (Example 43) in quantitative yield.

¹H-NMR (CDCl₃, 400 MHz): 12.33 (s br, 1H); 9.56 (s, 1H); 9.41 (s, 1H); 7.77 (s, 1H); 7.74 (t br, 1H); 7.21-7.38 (m, 7H); 6.91 (t br, 1H); 3.58 (s, 2H); 3.19-3.44 (m, 4H); 1.75-1.85 (m, 2H).

MS (ESI): [M−H]⁻=440.

### Intermediate 10

### Preparation of tert-Butyl N-methyl-N-[4-(4,4-dioxo-4-thia-2,5,9-triaza-1(2,4)-pyrimidina-3(1,3)-benzenacyclononaphan-1⁵-yl)phenyl]carbamate

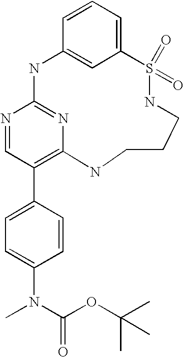

Intermediate 10 was prepared according to GP 1 from 1⁵-iodo-4-thia-2,5,9-triaza-1(2,4)-pyrimidina-3(1,3)-benzenacyclononaphan-4,4-dioxide and tert-butyl N-methyl-N-[4-(4,4,5,5-tetramethyl-1,3,2-dioxaborolan-2-yl)phenyl]carbamate.

¹H-NMR (DMSO, 400 MHz): 9.58 (s, 1H); 9.42 (s, 1H); 7.77 (s, 1H); 7.73 (t br, 1H); 7.31-7.38 (m, 5H); 7.26 (t br, 2H); 6.95 (t br, 1H); 3.32-3.44 (m, 2H); 3.20-3.28 (m, 2H); 3.18 (s, 3H); 1.74-1.87 (m, 2H); 1.38 (s, 9H). MS (ESI): [M+H]⁺=511.

### Preparation of Example Compounds

The following example compounds were prepared by Suzuki coupling according to the general procedure GP1 from 1⁵-iodo-4-thia-2,5,9-triaza-1(2,4)-pyrimidina-3(1,3)-benzenacyclononaphan-4,4-dioxide and the respective free boronic acid or in example 27 and 33 to 40 the corresponding pinacolate boronic esters.

| Example | Structure | | Analytical |
|---|---|---|---|
| No | And Yield | Name | Data |
| 1 | 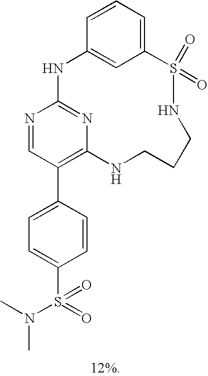 | 4-[4,4-dioxo-4λ⁶- thia-2,5,9-triaza- 1(2,4)-pyrimidina- 3(1,3)- benzenacyclonona- phan-1⁵-yl]-N,N- dimethylbenzene- sulfonamide | ¹H-NMR (DMSO, 300 MHz): 9.73 (s, 1 H); 9.43 (s, 1 H); 7.92 (s, 1 H); 7.75-7.83 (m, 3 H); 7.68 (d, 2 H); 7.38-7.46 (m, 1 H); 7.28-7.36 (m, 2 H); 7.19 (t br, 1 H); 3.22-3.46 (m, 4 H); 2.68 (s, 6 H); 1.78-1.94 (m, |
| # 2 H). MS (ESI): [M + H]⁺ = 489. | | | |
| 2 | 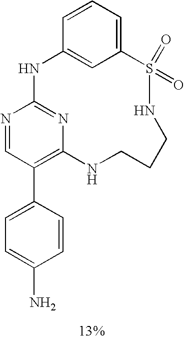 | 4-[4,4-dioxo-4λ⁶- thia-2,5,9-triaza- 1(2,4)-pyrimidina- 3(1,3)- benzenacyclonona- phan-1⁵-yl]- benzeneamine | ¹H-NMR (DMSO, 300 MHz): 9.50 (s, 1 H); 9.47 (s, 1 H); 7.76 (t, 1 H); 7.70 (s, 1 H); 7.32-7.41 (m, 1 H); 7.21-7.31 (m, 2 H); 7.03 (d, 2 H); 6.73 (t br, 1 H); 6.65 (d, 2 H); 5.18 (s br, 2 H); 3.19-3.50 (m, 4 H); 1.73-1.93 |
| # (m, 2 H). MS (ESI): [M + H]⁺ = 397. | | | |
| 3 | 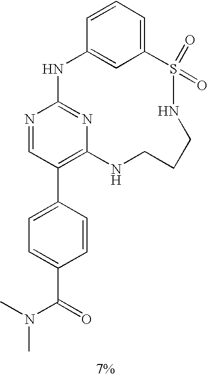 | 4-[4,4-dioxo-4λ⁶- thia-2,5,9-triaza- 1(2,4)-pyrimidina- 3(1,3)- benzenacyclonona- phan-1⁵-yl]-N,N- dimethylbenzene- amide | ¹H-NMR (DMSO, 400 MHz): 9.63 (s, 1 H); 9.45 (s, 1 H); 7.86 (s, 1 H); 7.77 (t, 1 H); 7.49 (mc, 4 H); 7.39 (d, 1 H); 7.27-7.37 (m, 2 H); 7.04 (t br, 1 H); 3.37-3.50 (m, 2 H); 3.23-3.30 (m, 2 H); 2.98 (s, 6 H); 1.77-1.92 (m, |
| # 2 H). MS (ESI): [M + H]⁺ = 453. | | | |
| 4 | 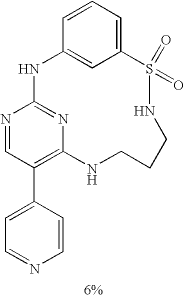 | 1⁵-(Pyridin-4-yl)-4- thia-2,5,9-triaza- 1(2,4)-pyrimidina- 3(1,3)- benzenacyclonona- phane-4,4-dioxide | ¹H-NMR (DMSO, 300 MHz): 9.73 (s, 1 H); 9.47 (s, 1 H); 8.62 (d, 2 H); 7.95 (s, 1 H); 7.78 (t, 1 H); 7.27-7.51 (m, 5 H); 7.22 (t, 1 H); 3.25-3.48 (m, 4 H); 1.74-1.92 (m, 2 H). MS (ESI): [M + H]⁺ = 383. |
| 5 | 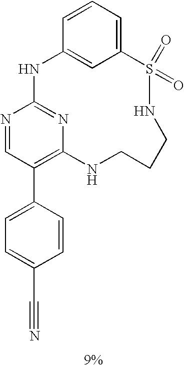 | 4-[4,4-dioxo-4λ⁶- thia-2,5,9-triaza- 1(2,4)-pyrimidina- 3(1,3)- benzenacyclonona- phan-1⁵-yl]- benzonitrile | ¹H-NMR (DMSO, 400 MHz): 9.70 (s, 1 H); 9.41 (s, 1 H); 7.83-7.92 (m, 3 H); 7.75 (t, 1 H); 7.57 (d, 2 H); 7.33-7.40 (m, 1 H); 7.24-7.31 (m, 2 H); 7.15 (t, 1 H); 3.30-3.45 (m, 2 H); 3.18-3.30 (m, 2 H); 1.73-1.88 |
| # (m, 2H). MS (ESI): [M + H]⁺ = 407. | | | |
| 6 | 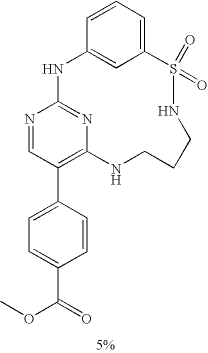 | methyl 4-[4,4-dioxo- 4λ⁶-thia-2,5,9-triaza- 1(2,4)-pyrimidina- 3(1,3)- benzenacyclonona- phan-1⁵-yl]benzoate | ¹H-NMR (DMSO, 300 MHz): 9.70 (s, 1 H); 9.44 (s, 1 H); 8.02 (d, 2 H); 7.89 (s, 1 H); 7.77 (t, 1 H); 7.57 (d, 2 H); 7.36-7.45 (m, 1 H); 7.25-7.34 (m, 2 H); 7.12 (t br, 1 H), 3.88 (s, 3 H); 3.35-3.48 (m, 2 H); 3.20-3.34 (m, |
| # 2 H); 1.70-1.95 (m, 2 H). MS (ESI): [M + H]⁺ = 440. | | | |
| 7 | 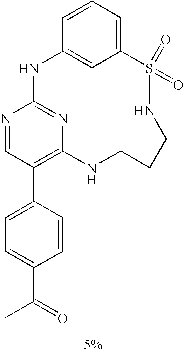 | 1-[4-[4,4-dioxo-4λ⁶- thia-2,5,9-triaza- 1(2,4)-pyrimidina- 3(1,3)- benzenacyclonona- phan-1⁵-yl]phenyl]- ethanone | ¹H-NMR (DMSO, 300 MHz): 9.71 (s, 1 H); 9.44 (s, 1 H); 8.03 (d, 2 H); 7.91 (s, 1 H); 7.78 (t, 1 H); 7.57 (d, 2 H); 7.37-7.47 (m, 1 H); 7.25-7.36 (m, 2 H); 7.12 (t, 1 H); 3.20-3.50 (m, 4 H); 2.63 (s, 3 H); 1.72-1.98 (m, 2 H). MS |
| # (ESI): [M + H]⁺ = 424. | | | |
| 8 | 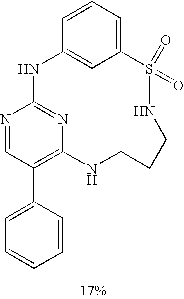 | 1⁵-phenyl-4-thia- 2,5,9-triaza-1(2,4)- pyrimidina-3(1,3)- benzenacyclonona- phane 4,4-dioxide | ¹H-NMR (DMSO, 300 MHz): 9.61 (s, 1 H); 9.47 (s, 1 H); 7.82 (s, 1 H); 7.78 (t, 1 H); 7.22-7.53 (m, 8 H); 6.96 (t, 1 H); 3.23-3.47 (m, 4 H); 1.76-1.96 (m, 2 H). MS (ESI): [M + H]⁺ = 382. |
| 9 | 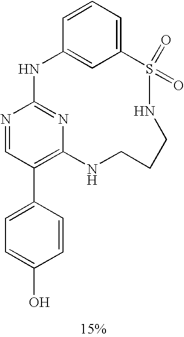 | 4-[4,4-dioxo-4λ⁶- thia-2,5,9-triaza- 1(2,4)-pyrimidina- 3(1,3)- benzenacyclonona- phan-1⁵-yl]phenol | ¹H-NMR (DMSO, 300 MHz): 9.53 (s, 1 H); 9.51 (s, 1 H); 9.47 (s, 1 H); 7.76 (t, 1 H); 7.72 (s, 1 H); 7.33-7.41 (m, 1 H); 7.24-7.32 (m, 2 H); 7.19 (d, 2 H); 6.87 (d, 2 H); 6.80 (t br, 1 H); 3.21-3.48 (m, 4 H); 1.73-1.93 (m, 2 H). MS (ESI): [M + H]⁺ = 398. |
| 10 | 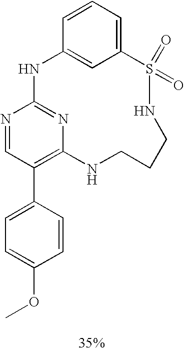 | 1⁵-(4- methoxyphenyl)-4- thia-2,5,9-triaza- 1(2,4)-pyrimidina- 3(1,3)- benzenacyclo- nonaphane 4,4- dioxide | ¹H-NMR (DMSO, 300 MHz): 9.58 (s, 1 H); 9.47 (s, 1 H); 7.68-7.83 (m, 2 H); 7.21-7.45 (m, 5 H); 7.02 (d, 2 H); 6.85 (t, 1 H); 3.79 (s, 3 H); 3.19-3.46 (m, 4 H); 1.72-1.95 (m, 2 H). MS (ESI): [M + H]⁺ = 412. |
| 11 | 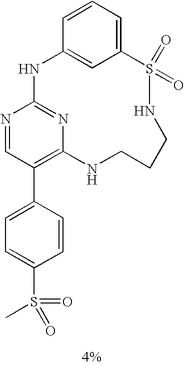 | 1⁵-(4- methylsulfonyl- phenyl)-4-thia-2,5,9- triaza-1(2,4)- pyrimidina-3(1,3)- benzenacyclonona- phane 4,4-dioxide | ¹H-NMR (DMSO, 300 MHz): 9.71 (s, 1 H); 9.46 (s, 1 H); 7.98 (d, 2 H); 7.88 (s, 1 H); 7.76 (s, br, 1 H); 7.67 (d, 2 H); 7.38-7.47 (m, 1 H); 7.28-7.36 (m, 2 H); 7.18 (t, 1 H); 3.24 (s, 3 H); 3.12-3.47 (m, 4 H); 1.76-1.94 (m, 2 H). MS (ESI): [M + H]⁺ = 460. |
| 12 | 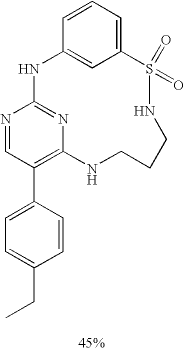 | 1⁵-(4-ethylphenyl)-4- thia-2,5,9-triaza- 1(2,4)-pyrimidina- 3(1,3)- benzenacyclonona- phane 4,4-dioxide | ¹H-NMR (DMSO, 300 MHz): 9.53 (s, 1 H); 9.43 (s br, 1 H); 7.67-7.77 (m, 2 H); 7.31-7.39 (m, 1 H); 7.20-7.29 (m, 6 H); 6.88 (t br, 1 H); 3.18-3.47 (m, 4 H); 2.61 (q, 2 H); 1.73-1.88 (m, 2 H); 1.17 (t, 3 H). MS (ESI): [M + H]⁺ = 410. |
| 13 | 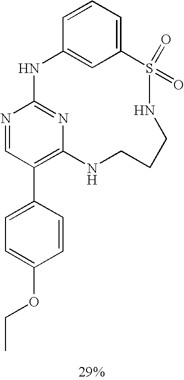 | 1⁵-(4-ethoxyphenyl)- 4-thia-2,5,9-triaza- 1(2,4)-pyrimidina- 3(1,3)- benzenacyclonona- phane 4,4-dioxide | ¹H-NMR (DMSO, 300 MHz): 9.52 (s, 1 H); 9.42 (s br, 1 H); 7.67-7.76 (m, 2 H); 7.70-7.78 (m, 1 H); 7.18-7.28 (m, 4 H); 6.96 (d, 2 H); 6.81 (t br, 1 H); 4.02 (q, 2 H); 3.18-3.43 (m, 4 H); 1.72-1.88 (m, 2 H); 1.30 (t, 3 H). MS (ESI): [M + H]⁺ = 426. |
| 14 | 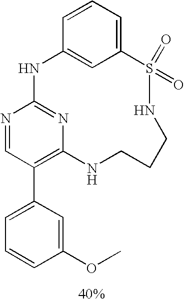 | 1⁵-(3- methoxyphenyl)-4- thia-2,5,9-triaza- 1(2,4)-pyrimidina- 3(1,3)- benzenacyclonona- phane 4,4-dioxide | ¹H-NMR (DMSO, 300 MHz): 9.59 (s, 1 H); 9.42 (s br, 1 H); 7.79 (s, 1 H); 7.73 (t br, 1 H); 7.21-7.40 (m, 4 H); 6.84-6.97 (m, 4 H); 3.74 (s, 3 H); 3.19-3.43 (m, 4 H); 1.72-1.88 (m, 2 H). MS (ESI): [M + H]⁺ = 412. |
| 15 | 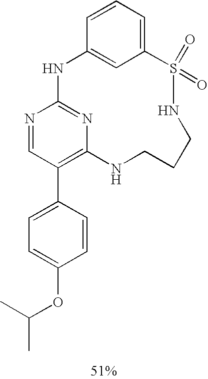 | 1⁵-(4-iso- propoxyphenyl)-4- thia-2,5,9-triaza- 1(2,4)-pyrimidina- 3(1,3)- benzenacyclonona- phane 4,4-dioxide | ¹H-NMR (DMSO, 300 MHz): 9.52 (s, 1 H); 9.42 (s br, 1 H); 7.67-7.75 (m, 2 H); 7.32-7.38 (m, 1 H); 7.20-7.30 (m, 4 H); 6.95 (d, 2 H); 6.82 (t br, 1 H); 4.60 (sept, 1 H); 3.18-3.42 (m, 4 H); 1.72-1.88 (m, 2 H); 1.26 (d, 6 H). MS (ESI): [M + H]⁺ = 440. |
| 16 | 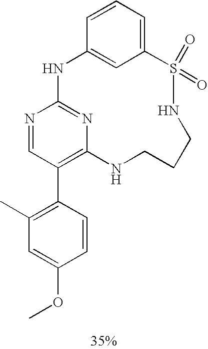 | 1⁵-(methoxy-2- methylphenyl)-4- thia-2,5,9-triaza- 1(2,4)-pyrimidina- 3(1,3)- benzenacyclonona- phane 4,4-dioxide | ¹H-NMR (DMSO, 400 MHz): 9.50 (s, 1 H); 9.43 (s, 1 H); 7.71 (t br, 1 H); 7.58 (s, 1 H); 7.34 (mc, 1 H); 7.24 (mc, 2 H); 7.01 (d, 1 H); 6.87 (d, 1 H); 6.80 (dd, 1 H); 6.41 (t br, 1 H); 3.73 (s, 3 H); 3.12-3.43 (m, 4 H); 2.05 (s, 3 H); 1.65-1.88 (m, 2 H). MS |
| # (ESI): [M + H]⁺ = 426. | | | |
| 17 | 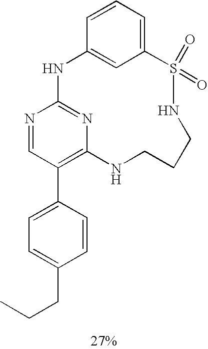 | 1⁵-(4-propylphenyl)- 4-thia-2,5,9-triaza- 1(2,4)-pyrimidina- 3(1,3)- benzenacyclonona- phane 4,4-dioxide | ¹H-NMR (DMSO, 400 MHz): 9.53 (s, 1 H); 9.41 (s br, 1 H); 7.68-7.78 (m, 2 H); 7.34 (mc, 1 H); 7.19-7.30 (m, 6 H); 6.87 (t br, 1 H); 3.31-3.45 (m, 2H); 3.18-3.29 (m, 2 H); 2.55 (mc, 2 H); 1.73-1.87 (m, 2 H); 1.59 (sext, 2 H); 0.89 (t, 3 H). MS (ESI): [M + H]⁺ = 424. |
| 18 | 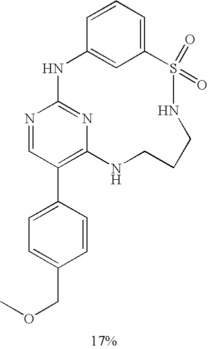 | 1⁵-[4- (methoxymethyl)- phenyl]-4-thia-2,5,9- triaza-1(2,4)- pyrimidina-3(1,3)- benzenacyclonona- phane 4,4-dioxide | ¹H-NMR (DMSO, 400 MHz): 9.58 (s, 1 H); 9.41 (s, 1 H); 7.78 (s, 1 H); 7.73 (t br, 1 H); 7.30-7.41 (m, 5 H); 7.25 (mc, 2 H); 6.92 (t br, 1 H); 4.41 (s, 2 H); 3.18-3.42 (m, 7 H); 1.72-1.90 (m, 2 H). MS (ESI): [M + H]⁺ = 426. |
| 19 | 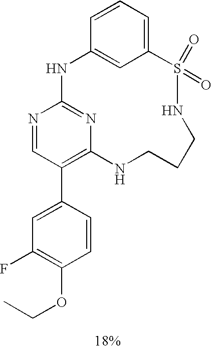 | 1⁵-(4-ethoxy-3- fluorophenyl)-4-thia- 2,5,9-triaza-1(2,4)- pyrimidina-3(1,3)- benzenacyclonona- phane 4,4-dioxide | ¹H-NMR (DMSO, 400 MHz): 9.57 (s, 1 H); 9.41 (s, 1 H); 7.68-7.77 (m, 2 H); 7.32-7.38 (m, 1 H); 7.15-7.30 (m, 4 H); 7.08 (d br, 1 H); 6.94 (t br, 1 H); 4.10 (q, 2 H); 3.32-3.41 (m, 2 H); 3.20-3.38 (m, 2 H); 1.72-1.86 (m, 2 H); 1.32 (t, 3 H). MS (ESI): [M + H]⁺ = 444. |
| 20 | 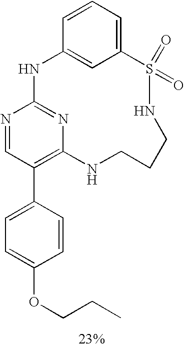 | 1⁵-(4- propoxyphenyl)-4- thia-2,5,9-triaza- 1(2,4)-pyrimidina- 3(1,3)- benzenacyclonona- phane 4,4-dioxide | ¹H-NMR (DMSO, 400 MHz): 9.52 (s, 1 H); 9.42 (s, 1 H); 7.68-7.75 (m, 2 H); 7.31-7.37 (m, 1 H); 7.21-7.28 (m, 4 H); 6.98 (d, 2 H); 6.81 (t br, 1 H); 3.92 (t, 2 H); 3.32-3.41 (m, 2 H); 3.18-3.28 (m, 2 H); 1.74-1.86 (m, 2 H); 1.72 (sext, 2 H); 0.96 (t, 3 H). MS |
| # (ESI): [M + H]⁺ = 440. | | | |
| 21 | 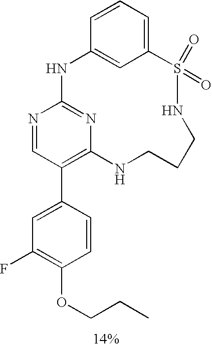 | 1⁵-(3-fluoro-4- propoxyphenyl)-4- thia-2,5,9-triaza- 1(2,4)-pyrimidina- 3(1,3)- benzenacyclonona- phane 4,4-dioxide | ¹H-NMR (DMSO, 400 MHz): 9.57 (s, 1 H); 9.41 (s, 1 H); 7.68-7.76 (m, 2 H); 7.72-7.78 (m, 1 H); 7.15-7.30 (m, 4 H); 7.08 (d br, 1 H); 6.92 (t br, 1 H); 4.00 (t, 2 H); 3.32-3.42 (m, 2 H); 3.20-3.28 (m, 2 H); 1.73-1.86 (m, 2 H); 1.72 (sext, 2 H); 0.95 (t, 3 H). MS |
| # (ESI): [M + H]⁺ = 458. | | | |
| 22 | 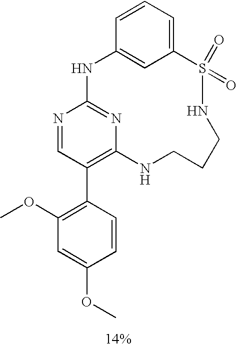 | 1⁵-(2,4- dimethoxyphenyl)- 4-thia-2,5,9-triaza- 1(2,4)-pyrimidina- 3(1,3)- benzenacyclonona- phane 4,4-dioxide | ¹H-NMR (DMSO, 400 MHz): 9.45 (s, 1 H); 9.42 (s, 1 H); 7.72 (t br, 1 H); 7.58 (s, 1 H); 7.30-7.37 (m, 1 H); 7.24 (mc, 2 H); 7.04 (d, 1 H); 6.61 (d, 1 H); 6.57 (dd, 1 H); 6.41 (t br, 1 H); 3.74 (s, 3 H); 3.68 (s, 3 H); 3.18-3.38 (m, 4 H); 1.71-1.84 (m, 2 H). MS |
| # (ESI): [M + H]⁺ = 442. | | | |
| 23 | 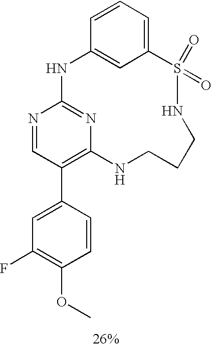 | 1⁵-(3-fluoro-4- methoxyphenyl)-4- thia-2,5,9-triaza- 1(2,4)-pyrimidina- 3(1,3)- benzenacyclonona- phane 4,4-dioxide | ¹H-NMR (DMSO, 400 MHz): 9.57 (s, 1 H); 9.42 (s, 1 H); 7.70-7.78 (m, 2 H); 7.32-7.38 (m, 1 H); 7.16-7.29 (m, 4 H); 7.11 (d br, 1 H); 6.94 (t br, 1 H); 3.83 (s, 3 H); 3.19-3.42 (m, 4 H); 1.72-1.87 (m, 2 H). MS (ESI): [M + H]⁺ = 430. |
| 24 | 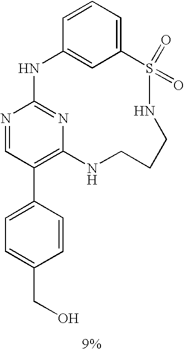 | 4-[(4,4-dioxo-4-thia- 2,5,9-triaza-1(2,4)- pyrimidina-3(1,3)- benzenacyclononaphan- 1⁵-yl)]benzene- methanol | ¹H-NMR (DMSO, 400 MHz): 9.57 (s, 1 H); 9.42 (s br, 1 H); 7.69-7.78 (m, 2 H); 7.22-7.42 (m, 7 H); 6.88 (t br, 1 H); 5.20 (t, 1 H); 4.50 (d, 2 H); 3.32-3.42 (m, 2 H); 3.19-3.28 (m, 2 H); 1.74-1.88 (m, 2 H). MS (ESI): [M + H]⁺ = 412. |
| 25 | 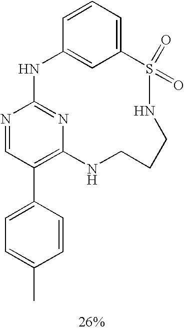 | 1⁵-(4-methylphenyl)- 4-thia-2,5,9-triaza- 1(2,4)-pyrimidina- 3(1,3)- benzenacyclonona- phane 4,4-dioxide | ¹H-NMR (DMSO, 400 MHz): 9.54 (s, 1 H); 9.42 (s, 1 H); 7.68-7.75 (m, 2 H); 7.34 (t, 1 H); 7.20-7.30 (m, 6 H); 6.85 (t br, 1 H); 3.31-3.43 (m, 2 H); 3.18-3.28 (m, 2 H); 2.32 (s, 3 H); 1.74-1.87 (m, 2 H). MS (ESI): [M + H]⁺ = 396. |
| 26 | 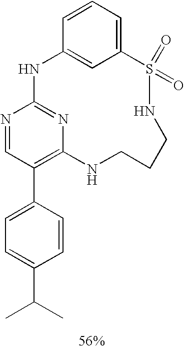 | 1⁵-(4-iso- propylphenyl)-4- thia-2,5,9-triaza- 1(2,4)-pyrimidina- 3(1,3)- benzenacyclonona- phane 4,4-dioxide | ¹H-NMR (DMSO, 400 MHz): 9.53 (s, 1 H); 9.42 (s, 1 H); 7.68-7.75 (m, 2 H); 7.21-7.38 (m, 7 H); 6.91 (t br, 1 H); 3.31-3.42 (m, 2 H); 3.19-3.28 (m, 2 H); 2.90 (sept, 1 H); 1.73-1.86 (m, 2 H); 1.18 (d, 6 H). MS (ESI): [M + H]⁺ = 424. |
| 27 | 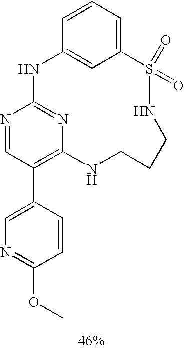 | 1⁵-(4-methoxypyrid- 3-yl)-4-thia-2,5,9- triaza-1(2,4)- pyrimidina-3(1,3)- benzenacyclonona- phane 4,4-dioxide | ¹H-NMR (DMSO, 300 MHz): 9.58 (s, 1 H); 9.42 (s, 1 H); 8.13 (d, 1 H); 7.70-7.78 (m, 2 H); 7.66 (dd, 1 H); 7.31-7.38 (m, 1 H); 7.21-7.29 (m, 2 H); 6.99 (t br, 1 H); 6.85 (d, 1 H); 3.83 (s, 3 H); 3.16-3.43 (m, 4 H), 1.69-1.90 (m, 2 H). MS (ESI): [M + H]⁺ = 413. |
| 28 | 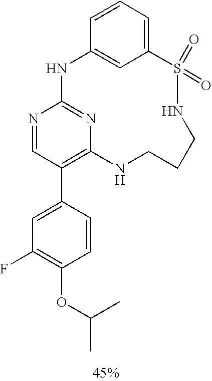 | 1⁵-(3-fluoro-4-iso- propoxyphenyl)-4- thia-2,5,9-triaza- 1(2,4)-pyrimidina- 3(1,3)- benzenacyclonona- phane 4,4-dioxide | ¹H-NMR (DMSO, 300 MHz): 9.56 (s, 1 H); 9.41 (s br, 1 H); 7.68-7.76 (m, 2 H); 7.30-7.38 (m, 1 H); 7.12-7.30 (m, 4 H); 7.07 (dd, 1 H); 6.93 (t br, 1 H); 4.62 (sept, 1 H); 3.18-3.42 (m, 4 H); 1.71-1.88 (m, 2 H); 1.28 (d, 6 H). MS (ESI): [M + H]⁺ = 458. |
| 29 | 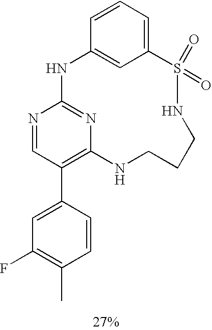 | 1⁵-(3-fluoro-4- methylphenyl)-4- thia-2,5,9-triaza- 1(2,4)-pyrimidina- 3(1,3)- benzenacyclonona- phane 4,4-dioxide | ¹H-NMR (DMSO, 300 MHz): 9.58 (s, 1 H); 9.41 (s, 1 H); 7.77 (s, 1 H); 7.73 (t br, 1 H); 7.21-7.38 (m, 5 H); 7.04-7.17 (m, 2 H), 6.97 (t br, 1 H); 3.17-3.43 (m, 4 H); 2.23 (s, 3 H); 1.72-1.88 (m, 2 H). MS (ESI): [M + H]⁺ = 414. |
| 30 | 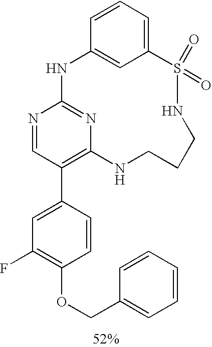 | 1⁵-[4-(benzyloxy)-3- fluorophenyl]-4-thia- 2,5,9-triaza-1(2,4)- pyrimidina-3(1,3)- benzenacyclonona- phane 4,4-dioxide | ¹H-NMR (DMSO, 300 MHz): 9.57 (s, 1 H); 9.41 (s, 1 H); 7.68-7.76 (m, 2 H); 7.18-7.60 (m, 10 H); 7.10 (d br, 1 H); 6.95 (t br, 1 H); 5.17 (s, 2 H); 3.17-3.43 (m, 4 H); 1.72-1.89 (m, 2 H). MS (ESI): [M + H]⁺ = 506. |
| 31 | 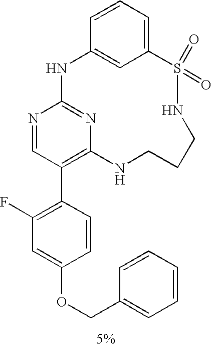 | 1⁵-[4-(benzyloxy)-2- fluorophenyl]-4-thia- 2,5,9-triaza-1(2,4)- pyrimidina-3(1,3)- benzenacyclonona- phane 4,4-dioxide | ¹H-NMR (DMSO, 400 MHz): 10.24 (s br, 1 H); 9.13 (s br, 1 H); 7.72-7.95 (m, 3 H); 7.31-7.48 (m, 8 H); 7.26 (t, 1 H); 7.03 (dd, 1 H); 6.96 (dd, 1 H); 5.14 (s, 2 H); 3.20-3.28 (m, 2 H); 3.30-3.40 (m, 2 H); 1.72-1.84 (m, 2 H). MS (ESI): [M + H]⁺ = 506. |
| 32 | 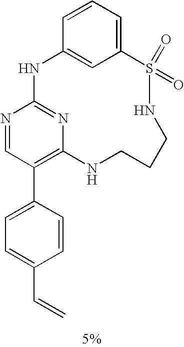 | 1⁵-(4-vinylphenyl)-4- thia-2,5,9-triaza- 1(2,4)-pyrimidina- 3(1,3)- benzenacyclonona- phane 4,4-dioxide | ¹H-NMR (DMSO, 400 MHz): 10.39 (s br, 1 H); 9.11 (s, 1 H); 8.02 (s br, 1 H); 7.82 (s, 1 H); 7.78 (t, 1 H); 7.59 (d, 2 H); 7.41-7.52 (m, 2 H); 7.28-7.39 (m, 3 H); 6.68 (dd, 1 H); 5.89 (d, 1 H); 5.30 (d, 1 H); 3.32-3.41 (m, 2 H); 3.19-3.28 (m, 2 H); 1.72-1.86 (m, 2 H). MS |
| # (ESI): [M + H]⁺ = 408. | | | |
| 33 | 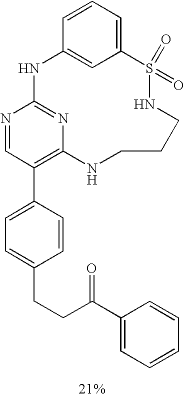 | 3-[4-(4,4-Dioxo-4- thia-2,5,9-triaza- 1(2,4)-pyrimidina- 3(1,3)- benzenacyclonona- phan-1⁵-yl)phenyl]- 1-phenylpropan-1- one | ¹H-NMR (DMSO, 400 MHz): 9.58 (s, 1 H); 9.44 (s, 1 H); 8.01 (d, 2 H); 7.78 (s, 1 H); 7.77 (t, 1 H); 7.67 (t, 1 H); 7.52 (t, 2 H); 7.25-7.42 (m, 7 H); 6.90 (t, 1 H); 3.60 (t, 2 H); 3.38-3.47 (m, 2 H); 3.25-3.33 (m, 2 H); 2.98 (t, 2 H); 1.80-1.90 (m, 2 H). MS |
| # (ESI): [M + H]⁺ = 514. | | | |
| 34 | 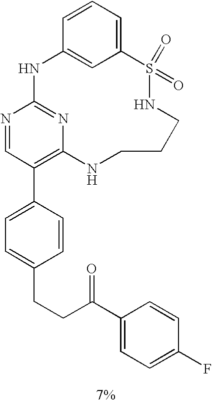 | 3-[4-(4,4-Dioxo-4- thia-2,5,9-triaza- 1(2,4)-pyrimidina- 3(1,3)- benzenacyclonona- phan-1⁵-yl)phenyl]- 1-(4-fluorophenyl)- propan-1-one | ¹H-NMR (DMSO, 400 MHz): 9.56 (s, 1 H); 9.41 (s, 1 H); 8.05 (dd, 2 H); 7.74 (s, 1 H); 7.72 (t, 1 H); 7.24-7.37 (m, 9 H); 6.90 (t, 1 H); 3.34-3.41 (m, 4 H); 3.20-3.26 (m, 2 H); 2.95 (t, 2 H); 1.76-1.83 (m, 2 H). MS (ESI): [M + H]⁺ = 532. |
| 35 | 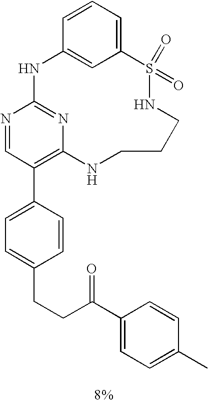 | 3-[4-(4,4-Dioxo-4- thia-2,5,9-triaza- 1(2,4)-pyrimidina- 3(1,3)- benzenacyclonona- phan-1⁵-yl)phenyl]- 1-(4-methylphenyl)- propan-1-one | ¹H-NMR (DMSO, 300 MHz): 9.54 (s, 1 H); 9.42 (s, 1 H); 7.91 (d, 2 H); 7.78 (s, 1 H); 7.77 (t, 1 H); 7.29-7.42 (m, 9 H); 6.90 (t, 1 H); 3.26-3.43 (m, 6 H); 2.96 (t, 2 H); 2.36 (s, 3 H); 1.80-1.92 (m, 2 H). MS (ESI): [M + H]⁺ = 528. |
| 36 | 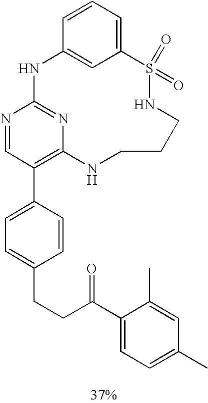 | 1-(2,4- Dimethylphenyl)-3- [4-(4,4-dioxo-4-thia- 2,5,9-triaza-1(2,4)- pyrimidina-3(1,3)- benzenacyclonona- phan-1⁵-yl)phenyl]- propan-1-one | ¹H-NMR (DMSO, 300 MHz): 9.55 (s, 1 H); 9.42 (s, 1 H); 7.73 (s, 1 H); 7.71-7.75 (m, 2 H); 7.23-7.37 (m, 7 H); 7.09 (d, 1 H); 7.08 (s, 1 H); 6.87 (t, 1 H), 3.33-3.40 (m, 2 H); 3.21-3.28 (m, 4 H); 2.91 (t, 2 H); 2.35 (s, 3 H); 2.26 (s, 3 H); |
| # 1.76-1.84 (m, 2 H). MS (ESI): [M + H]⁺ = 542. | | | |
| 37 | 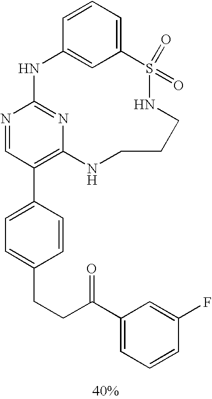 | 3-[4-(4,4-Dioxo-4- thia-2,5,9-triaza- 1(2,4)-pyrimidina- 3(1,3)- benzenacyclonona- phan-1⁵-yl)phenyl]- 1-(3-fluorophenyl)- propan-1-one | ¹H-NMR (DMSO, 300 MHz): 9.54 (s, 1 H); 9.42 (s, 1 H); 7.83 (d, 1 H); 7.76 (s, 1 H); 7.72-7.77 (m, 3 H); 7.56 (dt, 1 H); 7.45 (td, 1 H); 7.24-7.38 (m, 7 H); 6.86 (t, 1 H); 3.34-3.46 (m, 4 H); 3.20-3.27 (m, 2 H); 2.96 (t, 2 H); 1.74-1.86 (m, 2 H). MS |
| # (ESI): [M + H]⁺ = 532. | | | |
| 38 | 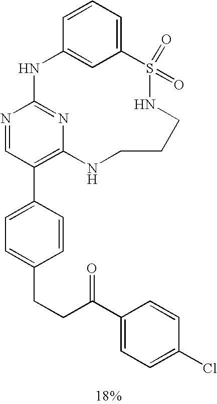 | 1-(4-Chlorophenyl)- 3-[4-(4,4-dioxo-4- thia-2,5,9-triaza- 1(2,4)-pyrimidina- 3(1,3)- benzenacyclonona- phan-1⁵-yl)phenyl]- propan-1-one | ¹H-NMR (DMSO, 400 MHz): 9.56 (s, 1 H); 9.43 (s, 1 H); 8.00 (d, 2 H); 7.75 (s, 1 H); 7.74 (t, 1 H); 7.57 (d, 2 H); 7.24-7.38 (m, 7 H); 6.87 (t, 1 H); 3.32-3.42 (m, 4 H); 3.20-3.26 (m, 2 H); 2.93 (t, 2 H); 1.74-1.84 (m, 2 H). MS (ESI): [M + |
| # H]⁺ = 548. | | | |
| 39 | 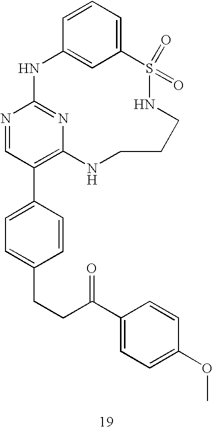 | 3-[4-(4,4-Dioxo-4- thia-2,5,9-triaza- 1(2,4)-pyrimidina- 3(1,3)- benzenacyclonona- phan-1⁵-yl)phenyl]- 1-(4-methoxy- phenyl)propan-1- one | ¹H-NMR (DMSO, 400 MHz): 10.10 (s, 1 H); 9.10 (s, 1 H); 7.95 (d, 2 H); 7.77 (t, 1 H); 7.76 (s, 1 H); 7.27-7.47 (m, 8 H); 7.00 (d, 2 H); 3.80 (s, 3 H); 3.34-3.50 (m, 4 H); 3.21-3.28 (m, 2 H); 2.95 (t, 2 H); 1.74-1.83 (m, 2 H). MS |
| # (ESI): [M + H]⁺ = 544. | | | |
| 40 | 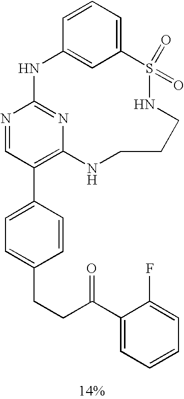 | 3-[4-(4,4-Dioxo-4- thia-2,5,9-triaza- 1(2,4)-pyrimidina- 3(1,3)- benzenacyclonona- phan-1⁵-yl)phenyl]- 1-(2-fluorophenyl)- propan-1-one | ¹H-NMR (DMSO, 300 MHz): 9.54 (s, 1 H); 9.42 (s, 1 H); 7.83 (td, 1 H); 7.74 (s, 1 H); 7.70-7.76 (m, 1 H); 7.60-7.65 (m, 1 H); 7.23-7.37 (m, 9 H); 6.88 (t, 1 H); 3.21-3.40 (m, 6 H); 2.94 (t, 2 H); 1.75-1.86 (m, 2 H). MS |
| # (ESI): [M + H]⁺ = 532. | | | |
| 41 | 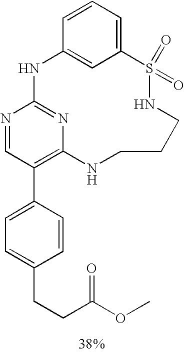 | Methyl 4-(4,4-dioxo- 4-thia-2,5,9-triaza- 1(2,4)-pyrimidina- 3(1,3)- benzenacyclonona- phan-1⁵-yl)benzene- propanoate | ¹H-NMR (CDCl₃, 300 MHz): 9.58 (s, 1 H); 9.45 (s, 1 H); 7.72-7.81 (m, 2 H); 7.22-7.43 (m, 7 H); 6.92 (t br, 1 H); 3.61 (s, 3 H); 3.22-3.47 (m, 4 H); 2.90 (t, 2 H); 2.67 (t, 2 H); 1.78-1.92 (m, 2 H). MS (ESI): [M − H]⁻ = 468. |
| 42 | 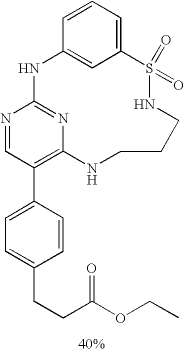 | Ethyl 4-(4,4-dioxo-4- thia-2,5,9-triaza- 1(2,4)-pyrimidina- 3(1,3)- benzenacyclonona- phan-1⁵-yl)benzene- propanoate | ¹H-NMR (CDCl₃, 300 MHz): 9.53 (s, 1 H); 9.41 (s, 1 H); 7.68-7.77 (m, 2 H); 7.21-7.38 (m, 7 H); 6.88 (t br, 1 H); 4.04 (q, 2 H); 3.18-3.43 (m, 4 H); 2.86 (t, 2 H); 2.61 (t, 2 H); 1.73-1.87 (m, 2 H); 1.13 (t, 3 H). MS (ESI): [M − H]⁻ = 482. |
| 43 | 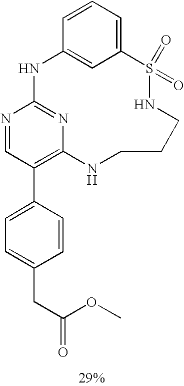 | Methyl 4-(4,4-dioxo- 4-thia-2,5,9-triaza- 1(2,4)-pyrimidina- 3(1,3)- benzenacyclonona- phan-1⁵-yl)benzene- acetate | ¹H-NMR (CDCl₃, 300 MHz): 9.60 (s, 1 H); 9.45 (s, 1 H); 7.81 (s, 1 H); 7.77 (t br, 1 H); 7.25-7.47 (m, 7 H); 6.96 (t br, 1 H); 3.73 (s, 2 H); 3.62 (s, 3 H); 3.22-3.48 (m, 4 H); 1.76-1.92 (m, 2 H). MS (ESI): [M − H]⁻ = 454. |

### Example 44

### Preparation of 1⁵-(thien-2-yl)-4-thia-2,5,9-triaza-1(2,4)-pyrimidina-3(1,3)-benzenacyclonona-phane 4,4-dioxide

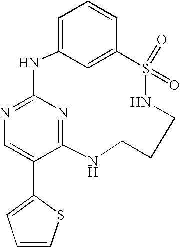

A suspension of 1⁵-iodo-4-thia-2,5,9-triaza-1(2,4)-pyrimidina-3(1,3)-benzenacyclononaphan-4,4-dioxide (150 mg; 0.36 mmol) and thiophene-2-boronic acid (65 mg, 0.51 mmol) in toluene/ethanol 1:1 (15 mL) was treated with aq. 1M Na₂CO₃ (0.9 mL), followed by LiCl (43 mg, 1.02 mmol) and Pd(PPh₃)₄ (32 mg, 0.03 mmol) and subsequently refluxed for 24 h. After cooling to ambient temperature, the mixture was diluted with ethyl acetate, filtered through Celite. The filtrate was washed with aq. NaHCO₃ and brine, dried over Na₂SO₄, and concentrated in vacuo. The crude residue was purified by flash column chromatography to give 37 mg of the desired compound (26%).

¹H-NMR (DMSO, 400 MHz): 9.68 (s, 1H); 9.41 (s, 1H); 7.89 (s, 1H); 7.74 (t, 1H); 7.51-7.57 (m, 1H); 7.34-7.41 (m, 1H); 7.25-7.31 (m, 2H); 7.11-7.18 (m, 2H); 7.06 (t, 1H); 3.35-3.49 (m, 2H); 3.20-3.30 (m, 2H); 1.75-1.91 (m, 2H). MS (ESI): [M+H]⁺=387.

### Example 45

### Preparation of 1⁵-(thien-3-yl)-4-thia-2,5,9-triaza-1(2,4)-pyrimidina-3(1,3)-benzenacyclonona-phane 4,4-dioxide

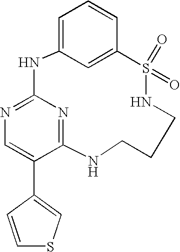

A suspension of 1⁵-iodo-4-thia-2,5,9-triaza-1(2,4)-pyrimidina-3(1,3)-benzenacyclononaphan-4,4-dioxide (150 mg; 0.36 mmol) and thiophene-3-boronic acid (65 mg, 0.51 mmol) in toluene/ethanol 1:1 (15 mL) was treated with aq. 1M Na₂CO₃ (0.9 mL), followed by LiCl (43 mg, 1.02 mmol) and Pd(PPh₃)₄ (32 mg, 0.03 mmol) and subsequently refluxed for 24 h. After cooling to ambient temperature, the mixture was diluted with ethyl acetate, filtered through Celite. The filtrate was washed with aq. NaHCO₃ and brine, dried over Na₂SO₄, and concentrated in vacuo. The crude residue was purified by flash column chromatography to give 65 mg of the desired compound (46%).

¹H-NMR (DMSO, 400 MHz): 9.63 (s, 1H); 9.45 (s, 1H); 7.93 (s, 1H); 7.78 (t, 1H); 7.64-7.71 (m, 1H); 7.52-7.60 (m, 1H); 7.34-7.41 (m, 1H); 7.21-7.31 (m, 3H); 6.94 (t, 1H); 3.36-3.51 (m, 2H); 3.23-3.34 (m, 2H); 1.75-1.92 (m, 2H).

MS (ESI): [M+H]⁺=387.

### Example 46

### Preparation of N-Benzyl-4-(4,4-dioxo-4-thia-2,5,9-triaza-1(2,4)-pyrimidina-3(1,3)-benzenacyclononaphan-1⁵-yl)benzeneacetamide

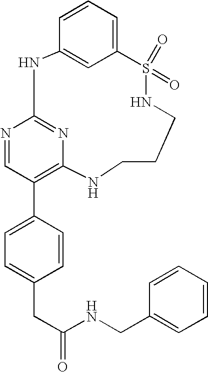

Example Compound 46 was prepared according to GP 3 from 4-(4,4-dioxo-4-thia-2,5,9-triaza-1(2,4)-pyrimidina-3(1,3)-benzenacyclononaphan-1⁵-yl)benzeneacetic acid (Intermediate 9) and benzylamine. Yield 15%.

¹H-NMR (DMSO, 300 MHz): 9.56 (s, 1H); 9.41 (s br, 1H); 8.57 (t br, 1H); 7.70-7.78 (m, 2H); 7.16-7.40 (m, 12H); 6.91 (t br, 1H); 4.24 (d, 2H); 3.49 (s, 2H); 3.18-3.45 (m, 4H); 1.72-1.88 (m, 2H).

MS (ESI): [M+H]⁺=529.

### Example 47

### Preparation of 4-(4,4-Dioxo-4-thia-2,5,9-triaza-1(2,4)-pyrimidina-3(1,3)-benzenacyclononaphan-1⁵-yl)-N-phenylbenzenepropanamide

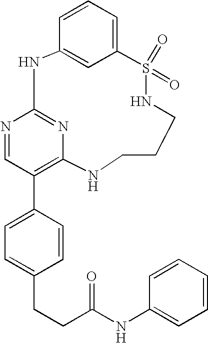

Example Compound 47 was prepared according to GP 3 from 4-(4,4-dioxo-4-thia-2,5,9-triaza-1(2,4)-pyrimidina-3(1,3)-benzenacyclononaphan-1⁵-yl)benzenepropanoic acid (Intermediate 8) and aniline. Yield 50%.

¹H-NMR (DMSO, 300 MHz): 9.91 (s, 1H); 9.53 (s, 1H); 9.41 (s, 1H); 7.68-7.80 (m, 2H); 7.57 (d, 2H); 7.20-7.40 (m, 10H); 6.98 (t, 1H); 6.89 (t br, 1H); 3.18-3.45 (m, 4H); 2.92 (t, 2H); 2.63 (t, 2H); 1.72-1.87 (m, 2H). MS (ESI): [M+H]+=529.

### Example 48

### Preparation of N-Benzyl-4-(4,4-dioxo-4-thia-2,5,9-triaza-1(2,4)-pyrimidina-3(1,3)-benzenacyctononaphan-1⁵-yl)benzenepropanamide

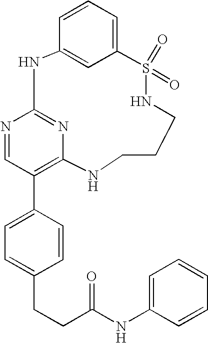

Example Compound 48 was prepared according to GP 3 from 4-(4,4-Dioxo-4-thia-2,5,9-triaza-1(2,4)-pyrimidina-3(1,3)-benzenacyclononaphan-1⁵-yl)benzenepropanoic acid (Intermediate 8) and benzylamine. Yield 46%.

¹H-NMR (DMSO, 300 MHz): 9.55 (s, 1H); 9.41 (s, 1H); 8.33 (t, 1H); 7.68-7.78 (m, 2H); 7.12-7.41 (m, 12H); 6.89 (t br, 1H); 4.27 (d, 2H); 3.16-3.44 (m, 4H); 2.86 (t, 2H); −2.47 (t, “2H”, mostly covered by DMSO peak); 1.72-1.87 (m, 2H).

MS (ESI): [M+H]⁺=544.

### Example 49

### Preparation of 4-(4,4-Dioxo-4-thia-2,5,9-triaza-1(2,4)-pyrimidina-3(1,3)-benzenacyclononaphan-1⁵-yl)-N-phenylbenzeneacetamide

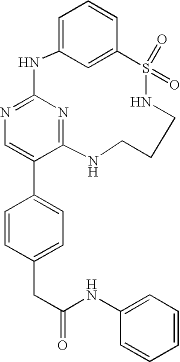

Example Compound 49 was prepared according to GP3 from 4-(4,4-dioxo-4-thia-2,5,9-triaza-1(2,4)-pyrimidina-3(1,3)-benzenacyclononaphan-1⁵-yl)benzeneacetic acid (Intermediate 9) and aniline. Yield 32%.

¹H-NMR (DMSO, 400 MHz): 10.18 (s, 1H); 9.56 (s, 1H); 9.41 (s, 1H); 7.75 (s, 1H); 7.72 (t, 1H); 7.58 (d, 2H); 7.40 (d, 2H); 7.22-7.37 (m, 7H); 7.01 (t, 1H); 6.91 (t br, 1H); 3.62 (s, 2H); 3.32-3.41 (m, 2H); 3.19-3.25 (m, 2H); 1.75-1.86 (m, 2H).

MS (ESI): [M+H]⁺=515.

### Example 50

### Preparation of 1⁵-ethynyl-4-thia-2,5,9-triaza-1(2,4)-pyrimidina-3(1,3)-benzenacyclonona-phane 4,4-dioxide

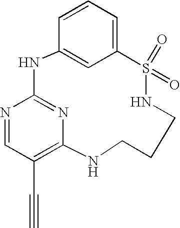 100 mg 1⁵-iodo-4-thia-2,5,9-triaza-1(2,4)-pyrimidina-3(1,3)-benzenacyclononaphan-4,4-dioxide (0.23 mmol), 8 mg PdCl₂(PPh₃)₂ (0.0115 mmol, 5 mol %) and 4 mg CuI (0.023 mmol, 10 mol %) were weighed into a Schlenk flask and placed under an atmosphere of argon. Dry DMF (2 mL) was added, followed by 0.64 mL triethylamine (4.6 mmol, 20 eq.) and 0.3 mL TMS-acetylene (1.15 mmol, 5 eq.). The resulting solution was stirred for 90 min, diluted with ethyl acetate and quenched with water. The aqueous layer was extracted with ethyl acetate, the combined organic layers dried and concentrated in vacuo. The residue was taken up in 3 mL THF and treated under argon with 0.3 mL of a TBAF solution (1.0 M in THF, 0.3 mmol). The resulting solution was stirred for 1 h at rt, quenched with water and extracted with ethyl acetate. The combined organic layers were dried and concentrated in vacuo. Flash column chromatography provided 26 mg (34%) of the desired alkyne as an off-white solid.

¹H-NMR (DMSO, 300 MHz): 9.80 (s, 1H); 9.35 (s, 1H); 8.02 (s, 1H); 7.75 (t, 1H); 7.20-7.38 (m, 4H); 4.41 (s, 1H); 3.35-3.43 (m, 2H); 3.20-3.30 (m, 2H); 1.75-1.88 (m, 2H). MS (ESI): [M−H]⁻=328.

### Examples 51 and 52

### Preparation of 1⁵-((Z)-styryl)-4-thia-2,5,9-triaza-1(2,4)-pyrimidina-3(1,3)-benzenacyclononaphane 4,4-dioxide (Example 51) and 1⁵-(1-phenyl-vinyl)-4-thia-2,5,9-triaza-1(2,4)-pyrimidina-3(1,3)-benzenacyclononaphane 4,4-dioxide (Example 52)

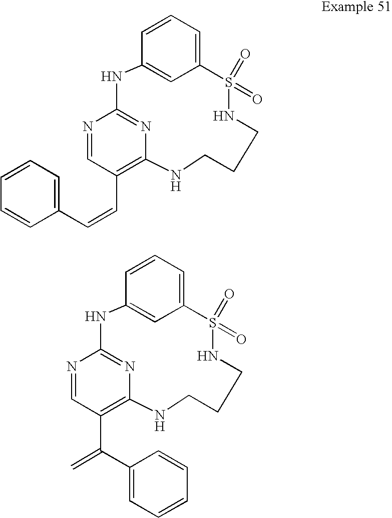

A suspension of 1⁵-iodo-4-thia-2,5,9-triaza-1(2,4)-pyrimidina-3(1,3)-benzenacyclononaphan-4,4-dioxide (165 mg, 0.38 mmol), palladium (II) acetate), (8.6 mg, 0.038 mmol), triphenylphosphine (20 mg, 0.08 mmol), potassium acetate (150 mg, 1.5 mmol), tetra-n-propylammonium bromide (101 mg, 0.38 mmol) and styrene (44 μL, 0.38 mmol) in DMF was stirred under argon at 70° C. for 7 h. Afterwards, the reaction mixture was poured into ice-cold aqueous sodium chloride solution. It was stirred for 8 h at 23° C. Then, the suspension was filtered and the crude precipitate was purified by column chromatography on silica gel.

The obtained mixture of example compounds 51 and 52 was separated by preparative HPLC (column: Xterra RP18, eluent: acetonitril, water, 0.1% NH₃) to yield 22 mg (14%) of Example Compound 51 and 4 mg (2.6%) of Example Compound 52.

### Example 51

¹H-NMR (DMSO): δ=9.55 (s, 1H); 9,44 (s, 1H); 7.79 (t, 1H); 7.64 (s, 1H); 7.15-7.40 (m, 9H); 6.00 (d, 1H); 6.31 (d, 1H); 3.60 (m, 2H); 3.40 (m, 2H);

1.82 (m, 2H).

MS (ESI): [M+H]⁺=408

### Example 52

¹H-NMR (DMSO): δ=9.63 (s, 1H); 9,42 (s, 1H); 7.73 (t, 1H); 7.70 (s, 1H); 7.25-7.42 (m, 9H); 5.82 (s, 1H); 5.33 (s, 1H); 3.61 (m, 2H); 3.40 (m, 2H); 1.74 (m, 2H).

MS (ESI): [M+H]⁺=408

### Example 53

### Preparation of 1⁵-(2-Phenylethynyl)-4-thia-2,5,9-triaza-1(2,4)-pyrimidina-3(1,3)-benzenacyclonona-phane 4,4-dioxide

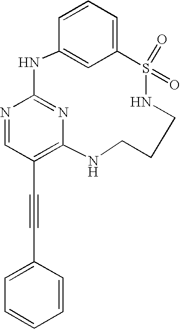

A suspension of 1⁵-iodo-4-thia-2,5,9-triaza-1(2,4)-pyrimidina-3(1,3)-benzenacyclononaphan-4,4-dioxide (200 mg; 0.46 mmol), phenylacetylene (611 μL; 5.56 mmol), copper (I) iodide (25 mg) and Pd(PPh₃)₄ (54 mg; 0.046 mmol) in Et₃N (20 ml) was stirred at 60° C. for 2.5 hours. Afterwards, the reaction mixture was poured into water. It was extracted with ethyl acetate. The organic layer was washed with brine, dried over sodium sulfate and evaporated in vacuo. The obtained crude material was purified by recrystallization from a mixture of dichloromethane/methanol (8:2). 175 mg (93%) product was isolated.

¹H-NMR (DMSO): δ=9.82 (s, 1H); 9.38 (s, 1H); 8.11 (s, 1H); 7.77 (t, 1H); 7.22-7.62 (m, 9H); 3.44 (m, 2H); 3.40 (m, 2H); 1.84 (m, 2H).

MS(ESI): [M+H]⁺=406

### Example 54

### Preparation of 1⁵-(2-Phenylethyl)-4-thia-2,5,9-triaza-1(2,4)-pyrimidina-3(1,3)-benzenacyclonona-phane 4,4-dioxide

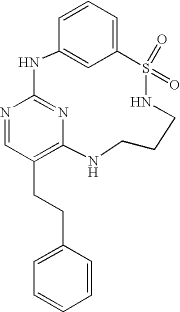

A solution of 1⁵-(2-phenylethynyl)-4-thia-2,5,9-triaza-1(2,4)-pyrimidina-3(1,3)-benzenacyclonona-phane 4,4-dioxide (100 mg; 0.25 mmol) and 10% palladium-on-charcoal (20 mg) in a mixture of tetrahydrofuran (5 mL) and ethanol (1 mL) was stirred under an atmosphere of hydrogen (ambient pressure) for 6 hours at 23° C. Then, it was filtered through Celite and evaporated in vacuum. The crude material was recrystallized from a mixture of dichloromethane/methanol (8:2). 66 mg (65%) product was isolated.

¹H-NMR (DMSO): δ=9.40 (s, 1H); 9.31 (s, 1H); 7.73 (t, 1H); 7.59 (s, 1H); 7.10-7.36 (m, 9H); 3.39 (m, 2H); 3.26 (m, 2H); 2.72 (t, 2H); 2.55 (t, 2H); 1.80 (m, 2H) ppm.

MS (ESI): [M+H]⁺=410

### Example 55

### Preparation of 4-[(4,4-dioxo-4-thia-2,5,9-triaza-1(2,4)-pyrimidina-3(1,3)-benzenacyclononaphan-1⁵-yl)]-N-methylbenzenamine

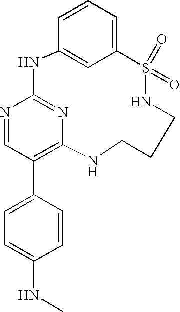

To a solution of Intermediate 10 (88 mg, 0.17 mmol) in acetonitrite (12 mL) was added 4 N HCL in dioxane (0.6 mL, 2.4 mmol), and the resulting mixture was stirred overnight at ambient temperature. Subsequently, another portion of 4 N HCl in dioxane (0.2 mL, 0.8 mmol) was added, and stirring at ambient temperature was continued for 3 h. The precipitate was isolated by filtration, washed with acetonitrite, and dissolved in water. By addition of aq. K₂CO₃, pH 10 was maintained, the mixture was stirred for 1 h at ambient temperature and the precipitated product was isolated by filtration, and dried to give the pure product (40 mg, 57%).

¹H-NMR (DMSO, 400 MHz): 9.47 (s, 1H); 9.43 (s, 1H); 7.73 (t br, 1H); 7.68 (s, 1H); 7.32 (t, 1H); 7.24 (mc, 2H); 7.07 (d, 2H); 6.72 (t br, 1H); 6.58 (d, 2H); 5.73 (q, 1H); 3.32-3.43 (m, 2H); 3.17-3.28 (m, 2H); 2.66 (d, 3H); 1.73-1.88 (m, 2H). MS (ESI): [M+H]+=411.

### Example 56

### Preparation of 1,1-Dimethylethyl-4-[(4,4-dioxo-4-thia-2,5,9-triaza-1(2,4)-pyrimidina-3(1,3)-benzenacyclononaphan-1⁵-yl)]piperazine-1-carboxylate

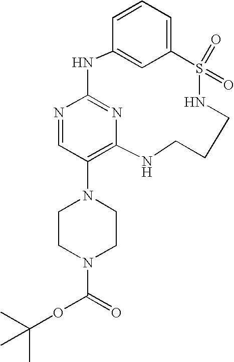

To a suspension of 1⁵-iodo-4-thia-2,5,9-triaza-1(2,4)-pyrimidina-3(1,3)-benzenacyclononaphan-4,4-dioxide (86 mg, 0.20 mmol) in DMSO (2 mL) was added tert-butyl piperazine carboxylate (186 mg, 1.00 mmol), copper(I) iodide (19 mg, 0.10 mmol), L-proline (23 mg, 0.20 mmol), and potassium carbonate (55 mg, 0.40 mmol). The resulting mixture was stirred under a nitrogen atmosphere for 6 h at 100° C. After cooling to ambient temperature, the mixture was concentrated in vacuo and the residue was triturated with water (5 mL), followed by HPLC purification (Purospher Star C18 5 μ25×150 mm, water (+0.2% NH₃)/acetonitrile gradient). Yield 13%.

¹H-NMR (DMSO, 400 MHz): 9.40 (s br, 1H); 9.34 (s, 1H); 7.72 (t, 1H); 7.68 (s, 1H); 7.32 (t, 1H); 7.17-7.24 (m, 2H); 7.03 (s br, 1H); 3.18-3.58 (m, 8H); 2.69 (s br, 4H); 1.74-1.87 (m, 2H); 1.38 (s, 9H). MS (ESI): [M+H]+=489.

### Example 57

### Preparation of 1⁵-(Morpholin-4-yl)-4-thia-2,5,9-triaza-1(2,4)-pyrimidina-3(1,3)-benzenacyclonona-phane 4,4-dioxide

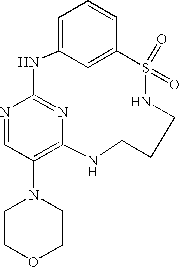

To a suspension of 1⁵-iodo-4-thia-2,5,9-triaza-1(2,4)-pyrimidina-3(1,3)-benzenacyclononaphan-4,4-dioxide (86 mg, 0.20 mmol) in DMSO (2 mL) was added morpholine (350 μL, 4.00 mmol), copper(I) iodide (19 mg, 0.10 mmol), L-proline (23 mg, 0.20 mmol), and potassium carbonate (55 mg, 0.40 mmol). The resulting mixture was stirred under a nitrogen atmosphere for 6 h at 100° C. After cooling to ambient temperature, the mixture was concentrated in vacuo and the residue was triturated with water (5 mL), followed by HPLC purification (Purospher Star C18 5 μ 25×150 mm, water (+0.2% NH₃)/acetonitrile gradient).

Yield 29%.

¹H-NMR (DMSO, 300 MHz): 9.38 (s br, 1H); 9.35 (s, 1H); 7.64-7.76 (m, 2H); 7.26-7.34 (m, 1H); 7.15-7.23 (m, 2H); 6.94 (t br, 1H); 3.68 (mc, 4H); 3.20-3.47 (m, 4H); 2.73 (mc, 4H); 1.72-1.89 (m, 2H). MS (ESI): [M+H]+=390. The following Example Compounds can be obtained using the methods described before or by standard procedures known to those skilled on the art:

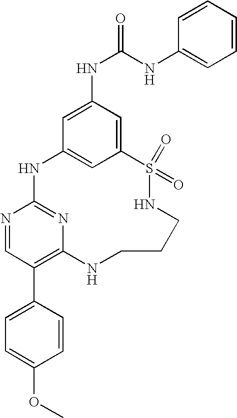

Example 58

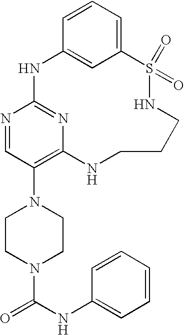

Example 59

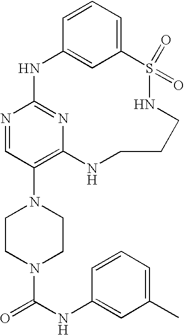

Example 60

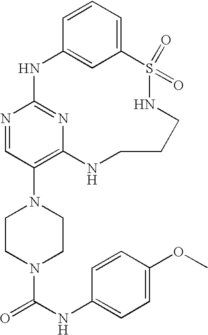

Example 61

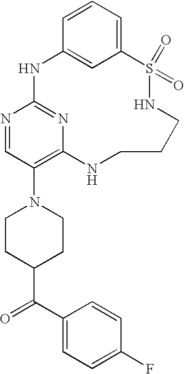

Example 62

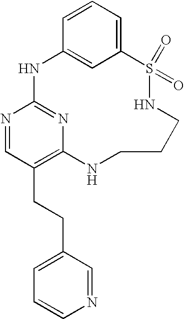

Example 63

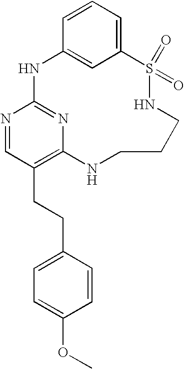

Example 64

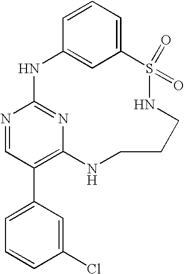

Example 65

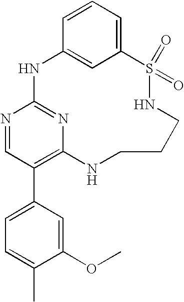

Example 66

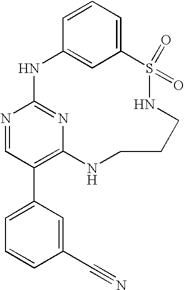

Example 67

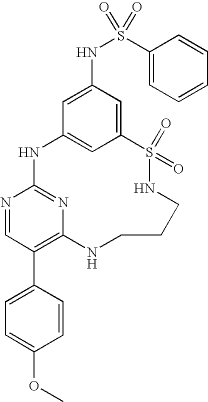

Example 68

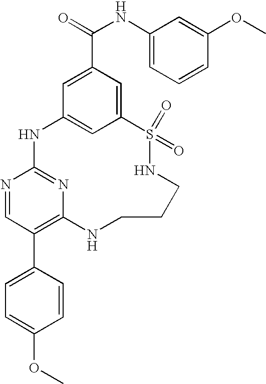

Example 69

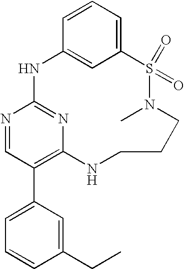

Example 70

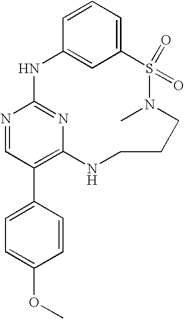

Example 71

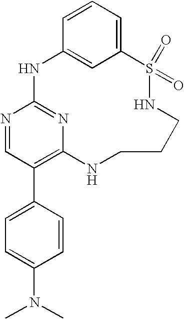

Example 72

Biological Experiment 1: ELISA Method

To prove the high potency activity as inhibitors of Tie2 kinase and Tie2 autophosphorylation the following ELISA-method was established and used.

Herein CHO cell-cultures, which are stably transfected by known techniques with Tie2 using DHFR deficiency as selection marker, are stimulated by angiopoietin-2. The specific autophosphorytation of Tie2 receptors is quantified with a sandwich-ELISA using anti-Tie2 antibodies for catch and anti-phosphotyrosine antibodies coupled to HRP as detection.

Materials:

96 well tissue culture plate, sterile, Greiner
96 well FluoroNunc plate MaxiSorp Surface C, Nunc
96 well plate polypropylene for compound dilution in DMSO
CHO Tie2/DHFR (transfected cells)
PBS−; PBS++, DMSO
MEM alpha Medium with Glutamax-I without Ribonucleosides and Deoxyribonucleosides (Gibco #32561-029)
with 10% FCS after dialysis! and 1% PenStrep
Lysis buffer: 1 Tablet “Complete” protease inhibitor
   1 cap Vanadate (1 mL>40 mg/mL; working solution 2 mM)
   ad 50 mL with Duschl-Puffer
   pH 7.6
Anti-TIE-II antibody 1:425 in Coating Buffer pH 9.6
   Stock solution: 1.275 mg/mL>working: 3 μg/mL
PBST: 2 bottles PBS(10×)+10 ml Tween, fill up with VE-water
RotiBlock 1:10 in VE-water
Anti-Phosphotyrosine HRP-Conjugated 1:10000 in 3% TopBlock
   3% TopBlock in PBST
BM Chemiluminescence ELISA Substrate (POD)
   solution B 1:100 solution A
SF9 cell culture medium
Ang2-Fc in SF9 cell culture medium
   Cell Experiment:
Dispense 5×10⁴ cells/well/98 μL in 96 well tissue culture plate
Incubate at 37° C./5% CO₂
After 24 h add compounds according to desired concentrations
Add also to control and stimulated values without compounds 2 μL DMSO
And mix for a few min at room temperature
Add 100 μL Ang2-Fc to all wells except control, which receives insect medium
Incubate 20 min at 37° C.
Wash 3× with PBS++
Add 100 μl Lysis buffer/well and shake a couple of min at room temperature
Store lysates at 20° C. before utilizing for the ELISA
   Performance of Sandwich-ELISA
Coat 96 well FluoroNunc Plate MaxiSorp Surface C with anti-Tie2 Mab
1:425 in Coating buffer pH 9.6; 100 μL/well overnight at 4° C.
Wash 2× with PBST
Blcock plates with 250 μL/well RotiBtock 1:10 in VE-water
Incubate for 2 h at room temperature or overnight at 4° C. shaking
Wash 2× in PBST
Add thawed lysates to wells and incubate overnight shaking at 4° C.
Wash 2× with PBST
Add 100 μL/well anti-Phosphotyrosine HRP-Conjugated 1:10000 in 3% TopBLock (3% TopBLock in PBST) and incubate overnight under shaking
Wash 6× with PBST
Add 100 μL/well BM Chemiluminescence ELISA Substrate (POD) solutions 1 und 2 (1:100)
Determine luminescence with the LumiCount.
   Biological Experiment 2: Tie-2-Kinase HTRF-Assay

To prove the effectiveness of the compound according to the present invention a Tie-2-Kinase HTRF-Assay was established.

Tie-2 phosphorylates tyrosine residues of the artificial substrate poLyGAT (biolinylated poLyGLuAlaTyr). Detection of phosphorylated product is achieved specifically by a trimeric detection complex consisting of the phosphorylated substrate, streptavidin-XLent (SA-XLent) which binds to biotin, and Europium Cryptate-LabeLed anti-phosphotyrosine antibody PT66 which binds to phosphorylated tyrosine. Excitation of Europium fluorescence with 337 nm light results in emission of long-lived light with 620 nm. In case a trimeric detection complex has formed, part of the energy will be transferred to the SA-XLent fluorophore that itself then emits long-lived light of 665 nm (FRET: fluorescence resonance energy transfer). Unphosphorylated substrate does not give rise to light emission at 665 nm, because no FRET-competent trimeric detection complex can be formed. Measurement is performed in a Packard Discovery or BMG Rubystar instrument. A-counts (emission at 665 nm) will be divided by B-counts (emission at 620 nm) and multiplicated with a factor of 10000. The resulting numbers are called the “well ratio” of the sample.

Material:

Enzyme: Tie-2-Kinase, in house, aliquots (12×10 mL) stored at −80° C.

Substrate: PoLyGAT labeled with Biotin (1000 μg/mL); CIS Bio; #61GATBLB; aliquots stored at −20° C.

ATP: Amersham Pharmacia Biotech Inc. #27-2056-01; 100 mM; stored at −20° C.
Antibody: PT66-Eu Cryptate; CIS Bio; # 61T66KLB; 30 μg/mL; aliquots stored at −20° C.
   SA-XLent; CIS Bio; #611SAXLB; 1000 μg/mL; aliquots stored at −80° C.
   Microplates: 384 Well black, SV, Greiner, #784076
   Solutions:
   Assay Buffer:
   50 mM HEPES (pH 7.0), 25 mM MgCl₂, 5 mM MnCl₂, 1 mM DTT, 0.5 mM Na₃VO₄, 0.01% (v/v) NP40, 1×Complete EDTA free
   Enzyme Working Solution:

Tie-2 stock solution is diluted 1:250 in assay buffer

Substrate Working Solution:

PolyGAT (1000 μg/mL; 36.23 μM) is diluted 1:90.6 to 400 nM or 77.3 ng/well, ATP (100 mM) is diluted 1:5000 to 20.0 μM. Both dilutions in assay buffer. Final assay concentrations: poly-GAT: 200 nM or 5.25 μg/mL, ATP: 10 μM (1×Km each).

Detection solution: 50 mM HEPES (pH 7.0), BSA 0.2%, 0.6 M KF, 200 mM EDTA, PT66-Europium Cryptate 2.5 ng/well, SA-XLent Cis Bio 90 ng/well.

Assay Steps

All steps at 20° C.

1. 0.75 μL of compound solution in 30% (v/v) DMSO

2. add 7 μL of substrate working solution

3. add 7 μL of enzyme working solution

4. incubate 75 min (reaction volume: 14.75 μL)

5. add 8 μL of detection solution

6. incubate 180 min or over night at 4° C. (total volume: 22.75 μL)

7. measure HTRF in Packard Discovery or BMG Rubystar instrument (delay 50 μs, integrated time 400 μs)

Final concentrations (in 14.75 μL reaction volume):

Enzyme: unknown

polyGAT (1×Km): 200 nM (77.3 ng)

ATP (1×Km): 10 μM

DMSO: 1.5% (v/v)

Buffer conditions: 50 mM HEPES (pH 7.0), 25 mM MgCl₂, 5 mM MnCl₂, 1 mM DTT, 0.5 mM NaVO4, 0.01% (v/v) NP40, 1×Complete

Controls:

C₀: uninhibited reaction (DMSO only)

Cᵢ: inhibited reaction with 20 μM Staurosporine

Biological Experiment 3: Proliferation Test

To examine cell toxicity a cell proliferation test were established.

With the cell proliferation test different tumour cell lines (e.g. Du 145) can be examined. The cells were dispensed in RPMI 1640 culture medium, supplied with 10% (v/v) fetal calf serum plus 1% (v/v) Penicillin/Streptomycin solution at a cell density of 2.000 cell/100 μL medium/per well (96 well plate). After three hours the cells were washed with PBS (containing calcium and magnesium). 100 μL of culture medium above with 0.1% (v/v) fetal calf serum was added and cultured at 37° C. and 5% CO₂-atmosphere. Next day compounds of the present invention diluted in DMSO for appropriate concentrations were added and further 100 μL culture medium 0.5% (v/v) fetal calf serum. After 5 days cell culturing at 37° C. and 5% CO₂-atmosphere cells were washed with PBS. 20 μL of glutaraldehyde solution (11% (v/v)) is added and the cells were slightly shaken at room temperature for 15 min. After that the cell were washed 3 times and dried in the air. 100 μL of crystal violet solution (0.1% at pH 3.5) were added and the cells were shaken for 30 min. The cells were washed with tap water and air-dried. The colour is dissolved with 100 μL of acetic acid (10% (v/v)) under strong shaking for 5 min. The absorption was measured at 595 nm wavelength.

The biological experiments show that the compounds presented in this application have high potency activity as inhibitors of Tie2 kinase and Tie2 autophosphorylation as measured with the ELISA-method. The IC50 values are below 1 μM. At the same time the toxicity of the compounds is well above 1 μM which is different to other compounds in this structure class, where the toxicity to tumour cell lines is such, that the IC50 values below 1 μM are observed.

Certain compounds of the invention have been found as potent inhibitors of Tie2. For example the synthesized example compounds 6, 7, 8, 9, 10, 11, 51 and 52 inhibit Tie2 with an IC50 of about 2 μM or less either in the Tie2 kinase assay or in the Tie2 autophosphorylation ELISA test. While featuring high inhibitory potency against Tie2 kinase activity, certain compounds of the invention have been found to be particularly weakly cytotoxic or non-cytotoxic. More specifically, selected example compounds 6, 7, 8 and 10 showed IC50>1 μM in the cytotoxicity assay using the cell line DU 145.

Biological Experiment 4: Tie-2 Kinase Assay Without Preactivation of Kinase

A recombinant fusion protein of GST and the intracellular domains of Tie-2, expressed in insect cells (Hi-5) and purified by Glutathion-Sepharose affinity chromatography was used as kinase. Alternatively, commercially available GST-Tie2-fusion protein (Upstate Biotechnology, Dundee, Scotland) can be used. As substrate for the kinase reaction the biolinylated peptide biotin-Ahx-EPKDDAYPLYSDFG (C-terminus in amid form) was used which can be purchased e.g. from the company Biosynthan GmbH (Berlin-Buch, Germany). Tie-2 (3.5 ng/measurement point) was incubated for 60 min at 22° C. in the presence of 10 μM adenosine-tri-phosphate (ATP) and 1 μM substrate peptide (biotin-Ahx-EPKDDAYPLYSDFG-NH₂) with different concentrations of test compounds (0 μM and concentrations in the range 0.001-20 μM) in 5 μl assay buffer [50 mM Hepes/NaOH pH 7, 10 mM MgCl₂, 0.5 mM MnCl₂, 1.0 mM dithiothreitol, 0.01% NP40, protease inhibitor mixture (“Complete w/o EDTA” from Roche, 1 tablet per 2.5 ml), 1% (v/v) dimethylsulfoxide]. The reaction was stopped by the addition of 5 μl of an aqueous buffer (25 mM Hepes/NaOH pH 7.5, 0.28% (w/v) bovine serum albumin) containing EDTA (90 mM) and the HTRF (Homogeneous Time Resolved Fluorescence) detection reagents streptavidine-XLent (0.2 μM, from Cis Biointernational, Marcoule, France) and PT66-Eu-Chelate (0.3 ng/μl; a europium-chelate labelled anti-phospho-tyrosine antibody from Perkin Elmer).

The resulting mixture was incubated 1 h at 22° C. to allow the binding of the biolinylated phosphorylated peptide to the streptavidine-XLent and the PT66-Eu-Chelate. Subsequently the amount of phosphorylated substrate peptide was evaluated by measurement of the resonance energy transfer from the PT66-Eu-Chelate to the streptavidine-XLent. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate peptide. The data were normalised (enzyme reaction without inhibitor=0% inhibition, all other assay components but no enzyme=100% inhibition) and IC₅₀ values were calculated by a 4 parameter fit using an inhouse software.

Biological Experiment 5: Tie-2 Kinase Assay with Preactivation of Kinase

A recombinant fusion protein of GST and the intracellular domains of Tie-2, expressed in insect cells (Hi-5) and purified by Glutathion-Sepharose affinity chromatography was used as kinase. As substrate for the kinase reaction the biolinylated peptide biotin-Ahx-EPKDDAYPLYSDFG (C-terminus in amid form) was used which can be purchased e.g. from the company Biosynthan GmbH (Berlin-Buch, Germany).

For activation, Tie-2 was incubated at a conc. 12.5 ng/μl of for 20 min at 22° C. in the presence of 250 μM adenosine-tri-phosphate (ATP) in assay buffer [50 mM Hepes/NaOH pH 7, 10 mM MgCl₂, 0.5 mM MnCl₂, 1.0 mM dithiothreitol, 0.01% NP40, protease inhibitor mixture (“Complete w/o EDTA” from Roche, 1 tablet per 2.5 ml)].

For the subsequent kinase reaction, the preactivated Tie-2 (0.5 ng/measurement point) was incubated for 20 min at 22° C. in the presence of 10 μM adenosine-tri-phosphate (ATP) and 1 μM substrate peptide (biotin-Ahx-EPKDDAYPLYSDFG-NH₂) with different concentrations of test compounds (0 μM and concentrations in the range 0.001-20 μM) in 5 μl assay buffer [50 mM Hepes/NaOH pH 7, 10 mM MgCl₂, 0.5 mM MnCl₂, 0.1 mM sodium ortho-vanadate, 1.0 mM dithiothreitol, 0.01% NP40, protease inhibitor mixture (“Complete w/o EDTA” from Roche, 1 tablet per 2.5 ml), 1% (v/v) dimethylsulfoxide]. The reaction was stopped by the addition of 5 p, of an aqueous buffer (25 mM Hepes/NaOH pH 7.5, 0.28% (w/v) bovine serum albumin) containing EDTA (90 mM) and the HTRF (Homogeneous Time Resolved Fluorescence) detection reagents streptavidine-XLent (0.2 μM, from Cis Biointernational, Marcoule, France) and PT66-Eu-Chelate (0.3 ng/μl; a europium-chelate labelled anti-phospho-tyrosine antibody from Perkin Elmer). The resulting mixture was incubated 1 h at 22° C. to allow the binding of the biolinylated phosphorylated peptide to the streptavidine-XLent and the PT66-Eu-Chelate. Subsequently the amount of phosphorylated substrate peptide was evaluated by measurement of the resonance energy transfer from the PT66-Eu-Chelate to the streptavidine-XLent. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate peptide. The data were normalised (enzyme reaction without inhibitor=0% inhibition, all other assay components but no enzyme=100% inhibition) and IC₅₀ values were calculated by a 4 parameter fit using an inhouse software.

The compounds of the present invention are therefore preferentially active as antiangiogenesis inhibitors and not as cytostatic or cytotoxic agents that affect tumour cells and other proliferating tissue cells directly.

1. A compound of the general Formula I: 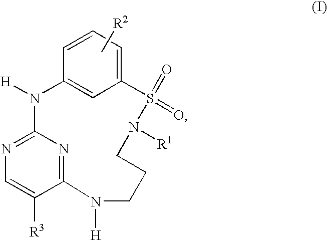
wherein
R¹ is hydrogen and —C₁-C₁₀-alkyl;
R² is selected from the group comprising, preferably consisting of, hydrogen, halogen, cyano, —NR⁴COR⁵, —NR⁴S(O)₂R⁵, —NR⁴CONR⁵R⁶, —NR⁴COOR⁵, —COR⁴, —S(O)₂R⁴, —S(O)₂NR⁴R⁵ and —CONR⁴R⁵;
R³ is selected from the group comprising, preferably consisting of, —C₁-C₆-alkyl, —C₂-C₆-alkenyl, —C₂-C₆-alkynyl, —C₃-C₈-cycloalkyl, —C₆-C₁₁-aryl and —C₅-C₁₀-heteroaryl, wherein said residues are unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, —C₂-C₆-alkenyl, —C₂-C₆-alkynyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃ and wherein one or more C atoms of the C-backbone of —C₃-C₈-cycloalkyl being optionally singly or multiply replaced independently from each other by nitrogen atoms, oxygen atoms, sulfur atoms and/or C═O-residues; or
optionally substituted phenyl-(CH₂)p-COR⁴ or optionally substituted phenyl-(CH₂)p-CONR⁴R⁵;
wherein p is an integer of 1 to 4, preferably 1 to 3, more preferably 1 or 2;
R⁴, R⁵, and R⁶ are the same or different and are independently from each other selected from the group comprising, preferably consisting of, hydrogen and residues being selected from the group comprising, preferably consisting of, —C₁-C₁₀-alkyl, —C₁-C₆-alkoxy, —C₂-C₆-alkenyl, —C₂-C₆-alkynyl, —C₃-C₈-cycloalkyl, —C₆-C₁₁-aryl and —C₅-C₁₀-heteroaryl, wherein said residues being unsubstituted or singly or multiply substituted independently from each other with hydroxy, halogen, —C₁-C₆-alkoxy, —C₁-C₆-alkylthio, amino, cyano, —C₁-C₆-alkyl, —C₃-C₁₀-cycloalkyl, —C₁-C₆-hydroxyalkyl, —C₂-C₆-alkenyl, —C₂-C₆-alkynyl, —C₁-C₆-alkoxy-C₁-C₆-alkyl, —NH—C₁-C₆-alkyl, —N(C₁-C₆-alkyl)₂, —S(O)(C₁-C₆-alkyl), —S(O)₂(C₁-C₆-alkyl), —C₁-C₆-alkanoyl, —CONR⁷R⁸, —COR⁷, carboxy, —C₆-C₁₁-aryl, —C₅-C₁₀-heteroaryl or —NR⁷R⁸; wherein —C₆-C₁-aryl and —C₅-C₁₀-heteroaryl being unsubstituted or singly or multiply substituted independently from each other with halogen, hydroxy, —C I—C₆-alkyl, —C₁-C₆-alkoxy, —CF₃ or —OCF₃ and wherein one or more C atoms of the C-backbone of —C₃-C₈-cycloalkyl being optionally singly or multiply replaced independently from each other by nitrogen atoms, oxygen atoms, sulfur atoms and/or C═O-residues;
R⁷ and R⁸ are the same or different and are independently from each other selected from the group comprising, preferably consisting of, —C₁-C₆-alkyl or phenyl, optionally substituted with halogen, hydroxy, —C₁-C₆-alkyl, —C₁-C₆-alkoxy, phenyl, —CF₃ and —OCF₃; and
solvates, hydrates, N-oxides, isomers, diastereomers, enantiomers and salts thereof.

2. The compound according to claim 1, wherein:
R³ is —C₁-C₆-alkyl being unsubstituted or singly or multiply substituted independently from each other with hydroxy, halogen, —C₁-C₆-alkoxy, —C₁-C₆-alkylthio, amino, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

3. The compound according to claim 1, wherein:
R³ is —C₂-C₆-alkenyl being unsubstituted or singly or multiply substituted independently from each other with hydroxy, halogen, —C₁-C₆-alkoxy, amino, —C₁-C₆-alkyl, —C₁-C₆-hydroxyalkyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

4. The compound according to claim 1, wherein:
R³ is —C₂-C₆-alkynyl being unsubstituted or singly or multiply substituted independently from each other with hydroxy, halogen, —C₁-C₆-alkoxy, amino, —C₁-C₆-alkyl, —C₁-C₆-hydroxyalkyl, —C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —CONR⁴R⁵, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃.

5. The compound according to claim 1, wherein:
R³ is —C₅-C₇-cycloalkyl being unsubstituted or singly or multiply substituted independently from each other with hydroxy, halogen, —C₁-C₆-alkyl, —S(O)₂(C₁-C₆-alkyl), —C₁-C₆-alkanoyl, —CONR⁴R⁵ and/or —COR⁴, wherein one or more C atoms of the C-backbone of-C₅-C₇-cycloalkyl being optionally replaced independently from each other by one or two nitrogen atoms.

6. The compound according to claim 5, wherein:
R³ is piperazine or piperidine, wherein piperazine or piperidine being unsubstituted or singly or multiply substituted independently from each other with —C₁-C₆-alkyl, —S(O)₂(C₁-C₆-alkyl), —C₁-C₆-alkanoyl, —CONR⁴R⁵ and/or —COR⁴.

7. The compound according to claim 1, wherein:
R³ is either:
—C₆-C₁₁-aryl being unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃;
or:
optionally substituted phenyl-(CH₂)ₚ—COR⁴ or optionally substituted phenyl-(CH₂)ₚ—CONR⁴R⁵,
wherein p is an integer of 1 to 3, preferably 1 or 2.

8. The compound according to claim 7, wherein:
R³ is either:
phenyl unsubstituted or singly or multiply substituted independently from each other with hydroxy, halogen, —C₁-C₆-alkoxy, —C₁-C₆-alkylthio, amino, —C₁-C₆-alkyl, —C₁-C₆-hydroxyalkyl, C₁-C₆-alkoxy-C₁-C₆-alkyl, —NH—C₁-C₆-alkyl, —N(C₁-C₆-alkyl)₂, —S(O)₂(C₁-C₆-alkyl), —C₁-C₆-alkanoyl, —COR⁴, —C₆-C₁₁-aryl, —C₅-C₁₀-heteroaryl and/or —NR⁴R⁵, wherein —C₆-C₁₁-aryl and —C₅-C₁₀-heteroaryl being unsubstituted or singly or multiply substituted independently from each other with halogen, hydroxy, —C₁-C₆-alkyl, —C₁-C₆-alkoxy, —CF₃ or —OCF₃;
or:
optionally substituted phenyl-(CH₂)ₚ—COR⁴ or optionally substituted phenyl-(CH₂)ₚ—CONR⁴R⁵,
wherein p is an integer of 1 to 3, preferably 1 or 2.

9. The compound according to claim 7, wherein:
R³ is either:
phenyl unsubstituted or singly or multiply substituted independently from each other with halogen, —C₁-C₆-alkoxy, —C₁-C₆-alkyl, —C₁-C₆-hydroxyalkyl, C₁-C₆-alkoxy-C₁-C₆-alkyl, —NH—C₁-C₆-alkyl, —N(C₁-C₆-alkyl)₂, —C₁-C₆-alkanoyl, —COR⁴, and/or —NR⁴R⁵;
or:
optionally substituted phenyl-(CH₂)ₚ—COR⁴ or optionally substituted phenyl-(CH₂)ₚ—CONR⁴R⁵,
wherein p is an integer of 1 or 2.

10. The compound according to claim 7, wherein:
R³ is phenyl singly or multiply substituted independently from each other with C₁-C₆-alkoxy, —C₁-C₆-alkyl.

11. The compound according to claim 7, wherein:
R³ is phenyl.

12. The compound according to claim 1, which is selected from the group consisting of:
4-[4,4-dioxo-4λ⁶-thia-2,5,9-triaza-1(2,4)-pyrimidina-3(1,3)-benzenacyclonona-phan-1⁵-yl]-N,N-dimethylbenzene-sulfonamide;
4-[4,4-dioxo-4λ-thia-2,5,9-triaza-1(2,4)-pyrimidina-3(1,3)-benzenacyclonona-phan-1⁵-yl]-benzeneamine;
4-[4,4-dioxo-4λ-thia-2,5,9-triaza-1(2,4)-pyrimidina-3(1,3)-benzenacyclonona-phan-1-yl]-dimethylbenzene-amide;
1⁵-(Pyridin-4-yl)-4-thia-2,5,9-triaza-1(2,4)-pyrimidina-3(1,3)-benzenacyclonona-phane-4,4-dioxide;
4-[4,4-dioxo-4λ⁶-thia-2,5,9-triaza-1(2,4)-pyrimidina-3(1,3)-benzenacyclonona-phan-1⁵-yl]-benzonitrile;
methyl 4-[4,4-dioxo-4λ⁶-thia-2,5,9-triaza-1(2,4)-pyrimidina-3(1,3)-benzena-cyclonona-phan-1⁵-yl]benzoate;
1-[4-[4,4-dioxo-4λ⁶-thia-2,5,9-triaza-1(2,4)-pyrimidina-3(1,3)-benzenacyclonona-phan-1⁵-yl]phenyl]-ethanone;
1⁵-phenyl-4-thia-2,5,9-triaza-1(2,4)-pyrimidina-3(1,3)-benzenacyclonona-phan-4,4-dioxide;
4-[4,4-dioxo-4λ⁶-thia-2,5,9-triaza-1(2,4)-pyrimidina-3(1,3)-benzenacyclonona-phan-1⁵-yl]phenol;
1⁵-(4-methoxyphenyl)-4-thia-2,5,9-triaza-1(2,4)-pyrimidina-3(1,3)-benzenacyclo-nonaphane 4,4-dioxide;
1⁵-(4-methylsulfonyl-phenyl)-4-thia-2,5,9-triaza-1(2,4)-pyrimidina-3(1,3)-benzenacyclonona-phane 4,4-dioxide;
1⁵-(4-ethylphenyl)-4-thia-2,5,9-triaza-1(2,4)-pyrimidina-3(1,3)-benzenacyclonona-phane 4,4-dioxide;
1⁵-(4-ethoxyphenyl)-4-thia-2,5,9-triaza-1(2,4)-pyrimidina-3(1,3)-benzenacyclonona-phane 4,4-dioxide;
1⁵-(3-methoxyphenyl)4-thia-2,5,9-triaza-1(2,4)-pyrimidina-3(1,3)-benzenacyclonona-phane 4,4-dioxide;
1⁵-(4-iso-propoxyphenyl)-4-thia-2,5,9-triaza-1(2,4)-pyrimidina-3(1,3)-benzenacyclonona-phane 4,4-dioxide;
1⁵-(4-methoxy-2-methylphenyl)-4-thia-2,5,9-triaza-1(2,4)-pyrimidina-3(1,3)-benzenacyclonona-phane 4,4-dioxide;
1⁵-(4-propylphenyl)-4-thia-2,5,9-triaza-1(2,4)-pyrimidina-3(1,3)-benzenacyclonona-phane 4,4-dioxide;
1⁵-[4-(methoxymethyl)-phenyl]-4-thia-2,5,9-triaza-1(2,4)-pyrimidina-3(1,3)-benze phane 4,4-dioxide;
1⁵-(4-ethoxy-3-fluorophenyl)-4-thia-2,5,9-triaza-1(2,4)-pyrimidina-3(1,3)-benzenacyclonona-phane 4,4-dioxide;
1⁵-(4-propoxyphenyl)-4-thia-2,5,9-triaza-1(2,4)-pyrimidina-3(1,3)-benzenacyclonona-phane 4,4-dioxide;
1⁵-(3-fluoro-4-propoxyphenyl)-4-thia-2,5,9-triaza-1(2,4)-pyrimidina-3(1,3)-benzenacyclonona-phane 4,4-dioxide;
1⁵-(2,4-dimethoxyphenyl)-4-thia-2,5,9-triaza-1(2,4)-pyrimidina-3(1,3)-benzenacyclonona-phane dioxide;
1⁵-(3-fluoro-4-methoxyphenyl)-4-thia-2,5,9-triaza-1(2,4)-pyrimidina-3(1,3)-benzenacyclonona-phane 4,4-dioxide;
4-[(4,4-dioxo-4-thia-2,5,9-triaza-1(2,4)-pyrimidina-3(1,3)-benzenacyclononaphan-1⁵-yl)]benzene-methanol;
1⁵-(4-methylphenyl)-4-thia-2,5,9-triaza-1(2,4)-pyrimidina-3(1,3)-benzenacyclonona-phane dioxide;
1⁵-(4-iso-propylphenyl)-4-thia-2,5,9-triaza-1(2,4)-pyrimidina-3(1,3)-benzenacyclonona-phane 4,4-dioxide;
1⁵-(4-methoxypyrid-3-yl)-4-thia-2,5,9-triaza-1(2,4)-pyrimidina-3(1,3)-benzenacyclonona-phane 4,4-dioxide;
1⁵-(3-fluoro-4-iso-propoxyphenyl)-4-thia-2,5,9-triaza-1(2,4)-pyrimidina-3(1,3)-benzenacyclonona-phane 4,4-dioxide;
1⁵-(3-fluoro-4-methylphenyl)-4-thia-2,5,9-triaza-1(2,4)-pyrimidina-3(1,3)-benzenacyclonona-phane 4,4-dioxide;
1⁵-[4-(benzyloxy)-3-fluorophenyl]-4-thia-2,5,9-triaza-1(2,4)-pyrimidina-3(1,3)-benzenacyclonona-phane 4,4-dioxide;
1⁵-[4-(benzyloxy)-2-fluorophenyl]-4-thia-2,5,9-triaza-1(2,4)-pyrimidina-3(1,3)-benzenacyclonona-phane 4,4-dioxide;
1⁵-(4-vinylphenyl)-4-thia-2,5,9-triaza-1(2,4)-pyrimidina-3(1,3)-benzenacyclonona-phane 4,4-dioxide;
3-[4-(4,4-Dioxo-4-thia-2,5,9-triaza-1(2,4)-pyrimidina-3(1,3)-benzenacyclonona-phan-1⁵-yl)phenyl]-1-phenylpropan-1-one;
3-[4-(4,4-Dioxo-4-thia-2,5,9-triaza-1(2,4)-pyrimidina-3(1,3)-benzenacyclonona-phan-1⁵-yl)phenyl]-1-(4-fluorophenyl)propan-1-one;
3-[4-(4,4-Dioxo-4-thia-2,5,9-triaza-1(2,4)-pyrimidina-3(1,3)-benzenacyclonona-phan-1⁵-yl)phenyl]-1-(4-methylphenyl)-propan-1-one;
1-(2,4-Dimethylphenyl)-3-[4-(4,4-dioxo-4-thia-2,5,9-triaza-1(2,4)-pyrimidina-3 benzenacyclonona-phan-1⁵-yl)phenyl]-propan-1-one;
3-[4-(4,4-Dioxo-4-thia-2,5,9-triaza-1(2,4)-pyrimidina-3(1,3)-benzenacyclonona-phan-1⁵-yl)phenyl]-1-(3-fluorophenyl)propan-1-one;
1-(4-Chlorophenyl)-3-[4-(4,4-dioxo-4-thia-2,5,9-triaza-1(2,4)-pyrimidina-3(1,3)-benzenacyclonona-phan-1⁵-yl)phenyl]-propan-1-one;
3-[4-(4,4-Dioxo-4-thia-2,5,9-triaza-1(2,4)-pyrimidina-3(1,3)-benzenacyclonona-phan-1⁵-yl)phenyl]-1-(4-methoxy-phenyl)propan-1-one;
3-[4-(4,4-Dioxo-4-thia-2,5,9-triaza-1(2,4)-pyrimidina-3(1,3)-benzenacyclonona-phan-1⁵-yl)phenyl]-1-(2-fluorophenyl)propan-1-one;
Methyl 4-(4,4-dioxo-4-thia-2,5,9-triaza-1(2,4)-pyrimidina-3(1,3)-benzenacyclonona-phan-1⁵-yl)benzene-propanoate;
Ethyl 4-(4,4-dioxo-4-thia-2,5,9-triaza-1(2,4)-pyrimidina-3(1,3)-benzena-cyclonona-phan-1⁵-yl)benzene-propanoate;
Methyl 4-(4,4-dioxo-4-thia-2,5,9-triaza-1(2,4)-pyrimidina-3(1,3)-benzena-cyclonona-phan-1⁵-yl)benzene-acetate;
1⁵-(thien-2-yl)-4-thia-2,5,9-triaza-1(2,4)-pyrimidina-3(1,3)-benzenacyclonona-phane 4,4-dioxide;
1⁵-(thien-3-yl)-4-thia-2,5,9-triaza-1(2,4)-pyrimidina-3(1,3)-benzenacyclonona-phane 4,4-dioxide;
N-Benzyl-4-(4,4-dioxo-4-thia-2,5,9-triaza-1(2,4)-pyrimidina-3(1,3)-benzenacyclononaphan-1⁵-yl)benzeneacetamide;
4-(4,4-Dioxo-4-thia-2,5,9-triaza-1(2,4)-pyrimidina-3(1,3)-benzenacyclononaphan-1⁵-yl-N-phenylbenzenepropanamide;
N-Benzyl-4-(4,4-dioxo-4-thia-2,5,9-triaza-1(2,4)-pyrimidina-3(1,3)-benzenacyclononaphan-1⁵-yl)benzenepropanamide;
4-(4,4-Dioxo-4-thia-2,5,9-triaza-1(2,4)-pyrimidina-3(1,3)-benzenacyclononaphan-1⁵-yl-N-phenylbenzeneacetamide;
1⁵-ethynyl-4-thia-2,5,9-triaza-1(2,4)-pyrimidina-3(1,3)-benzenacyclonona-phane 4,4-dioxide;
1⁵-((Z)-styryl)-4-thia-2,5,9-triaza-1(2,4)-pyrimidina-3(1,3)-benzenacyclononaphane 4,4-dioxide;
1⁵-(1-phenyl-vinyl)-4-thia-2,5,9-triaza-1(2,4)-pyrimidina-3(1,3)-benzenacyclononaphane 4,4-dioxide;
1⁵-(2-Phenylethynyl)-4-thia-2,5,9-triaza-1(2,4)-pyrimidina-3(1,3)-benzenacyclonona-phane 4,4-dioxide;
1⁵-(2-Phenylethyl)-4-thia-2,5,9-triaza-1(2,4)-pyrimidina-3(1,3)-benzenacyclonona-phane 4,4-dioxide;
4-[(4,4-dioxo-4-thia-2,5,9-triaza-1(2,4)-pyrimidina-3(1,3)-benzenacyclononaphan-1⁵-yl)]-N-methylbenzenamine;
1,1-Dimethylethyl-4-[(4,4-dioxo-4-thia-2,5,9-triaza-1(2,4)-pyrimidina-3(1,3)-benzenacyclononaphan-1⁵-yl)]piperazine-1-carboxylate; and
1⁵-(Morpholin-4-yl)₄-thia-2,5,9-triaza-1(2,4)-pyrimidina-3(1,3)-benzenacyclonona-phane 4,4-dioxide;

13. A method of preparing the compound according to claim 1, wherein the method comprises the following method step: 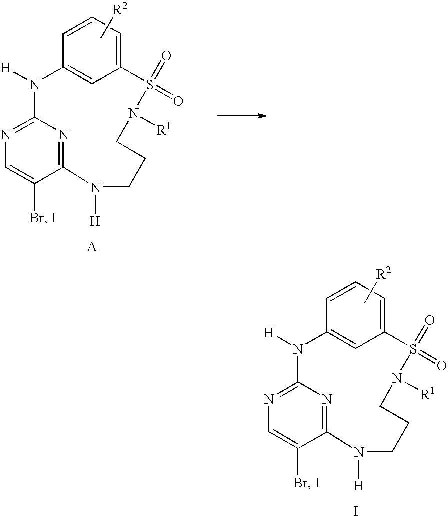
wherein R¹ and R² in the halogenated macrocycle A have the same meaning as in Formula I, wherein in Formula I R³ is selected from the group comprising unsubstituted or substituted ethyl, vinyl, ethynyl, phenyl or heteroaryl with the meaning above.

14. A method of preparing the compound according to claim 1, wherein the method comprises the following method step: 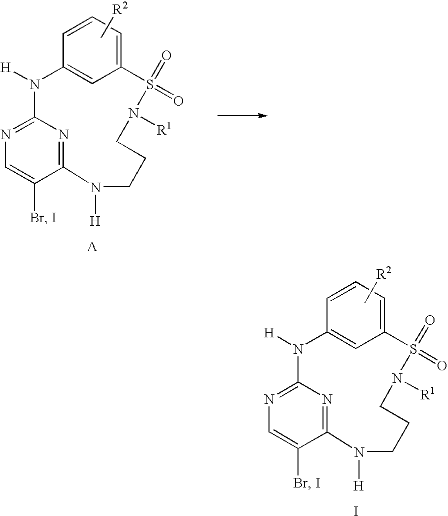
wherein R¹ and R² in the halogenated macrocycle A have the same meaning as in Formula I, wherein in Formula I R³ is unsubstituted or substituted —C₃-C₁₀-cycloalkyl containing at least one hetero atom, replaces the halide in the macrocycle A.

15. A pharmaceutical composition which comprises the compounds of the Formula I according to claim 1 or a pharmaceutically acceptable salt or an in vivo hydrolysable ester thereof, and a pharmaceutically-acceptable diluent or carrier.

16. A use of the compound of claim 1 for manufacturing a pharmaceutical composition for the treatment of diseases of dysregulated vascular growth or of diseases which are accompanied with dysregulated vascular growth.

17. The use according to claim 16, wherein said diseases are retinopathy, other angiogenesis dependent diseases of the eye, rheumatoid arthritis, and other inflammatory diseases associated with angiogenesis.

18. The use of claim 17, wherein said angiogenesis dependent diseases of the eye are cornea transplant rejection, age-related macular degeneration.

19. The use of claim 17, wherein said inflammatory diseases associated with angiogenesis are psoriasis, delayed type hypersensitivity, contact dermatitis, asthma, multiple sclerosis, restenosis, pulmonary hypertension, stroke, and diseases of the bowel.

20. The use according to claim 16, wherein said diseases are coronary and peripheral artery disease.

21. The use according to claim 16, wherein said diseases are ascites, oedema such as brain tumour associated oedema, high altitude trauma, hypoxia induced cerebral oedema pulmonary oedema and macular oedema or oedema following burns and trauma, chronic lung disease, adult respiratory distress syndrome, bone resorbtion and for benign proliferating diseases such as myoma, benign prostate hyperplasia and wound healing for the reduction of scar formation, reduction of scar formation scar formation during regeneration of damaged nerves, endometriosis, pre-eclampsia, postmenopausal bleeding and ovarian hyperstimulation.

22. The use according to claim 16, wherein the diseases is a solid tumour and/or metastases thereof.

23. A compound of formula (I) according to claim 1 for use as an inhibitor of the kinase Tie2.

24. A method for treating a disease of dysregulated vascular growth or diseases which are accompanied with dysregulated vascular growth by using of the compound of claim 1.

25. The method according to claim 24, wherein the diseases are retinopathy, other angiogenesis dependent diseases of the eye rheumatoid arthritis, and other inflammatory diseases associated with angiogenesis.

26. The method according to claim 25, wherein the angiogenesis dependent diseases of the eye are cornea transplant rejection, age-related macular degeneration.

27. The method according to claim 25, wherein the inflammatory diseases associated with angiogenesis are psoriasis, delayed type hypersensitivity, contact dermatitis, asthma, multiple sclerosis, restenosis, pulmonary hypertension, stroke, and diseases of the bowel.

28. The method according to claim 24, wherein the diseases are coronary and peripheral artery disease.

29. The method according to claim 24, wherein the diseases are ascites, oedema such as brain tumour associated oedema, high altitude trauma, hypoxia induced cerebral oedema pulmonary oedema and macular oedema or oedema following burns and trauma, chronic lung disease, adult respiratory distress syndrome, bone resorbtion and for benign proliferating diseases such as myoma, benign prostate hyperplasia and wound healing for the reduction of scar formation, reduction of scar formation scar formation during regeneration of damaged nerves, endometriosis, pre-eclampsia, postmenopausal bleeding and ovarian hyperstimulation.

30. The method according to claim 24, wherein the diseases is a solid tumour and/or metastases thereof.
